(19) United States
(12) Patent Application Publication
(10) Pub. No.: US 2018/0185827 A1
(43) Pub. Date: Jul. 5, 2018

(54) Mesoporous Zeolitic Material, Method for Making the Same and Use

(71) Applicants: Total Research & Technology Feluy, Seneffe (Feluy) (BE); Centre National de la Recherche Scientifique (CNRS, Paris (FR)

(72) Inventors: Xiaowei Cheng, Shanghai (CN); Corine Gerardin, Grabels (FR); François Fajula, Teyran (FR); Laura Vaugon, Beillargu (FR); Delphine Minoux, Nivelles (BE); Jean-Pierre Dath, Beloeil (BE)

(21) Appl. No.: 15/741,520

(22) PCT Filed: Jul. 5, 2016
(86) PCT No.: PCT/EP2016/065764

§ 371 Date: Jan. 3, 2018

(30) Foreign Application Priority Data
Jul. 9, 2015 (EP) 15290179.9

(51) Int. Cl. B01J 29/65; C01B 39/44; B01J 37/00; B01J 37/30; C07C 2/12

(52) U.S. Cl. CPC B01J 29/65; C01B 39/44; C01B 39/445; B01J 37/0018; C07C 2529/65; C07C 2/12; B01J 2229/38; B01J 2229/126; B01J 37/30

(57) ABSTRACT

A mesoporous zeolitic material possessing an ordered mono-dimensional (1D) or two-dimensional (2D) network of micropores (ie pores<2 nm in diameter) containing mesopores (pores with diameters in the range 2-50 nm) connected to the microporores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores.

### SUMMARY DESCRIPTION OF THE MAIN FEATURES OF THE INVENTION

A material possessing an ordered uni-directional (1D) or two-dimensional (2D) network of micropores (ie pores<2 nm in diameter) containing mesopores (pores with diameters in the range 2-50 nm) connected to the microporores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores.

### Advantages of the Invention

Generation of an oriented network of mesopores with adjustable sizes in a microporous material. The mesopores are oriented in the directions of the micropores and connected to the external surface by restrictions. The material is useful for traditional catalysis, sorption, separation, ion-exchange applications as well as for advanced applications based on occlusion chemistry.

### STATE OF THE ART

The zeolite ferrierite (also named ZSM-35; FER-type), displays unique textural properties advantageously used in some acid-catalyzed reactions, such as skeletal isomerization of n-alkenes to iso-alkenes (J. Catal., 2009, 265, 170-180 and J. Catal. 2002, 211, 366-378), propene oxidative dehydrogenation (Appl. Catal. A 2002, 235, 181-191), NOₓ reduction (1998, Chem. Commun. 2755-2756), epoxidation of styrene (Catal. Lett. 1999, 62, 209-213), etc. The micropore (<2 nm) architecture of ferrierite, is characterized by 10-membered ring (10-MR) (0.42×0.54 nm, along [001] direction) channels perpendicularly intersected by 8-MR (0.35×0.48 nm, along [010] direction) channels in framework (U.S. Pat. No. 4,016,245).

Ferrierite generally appears as plate-like crystals (J. Catal., 2009, 265, 170-180). The 8- or 10-MR channels, which support the main diffusion path for molecules, run parallel to the plate. 6-MR channels exist along the [101] direction (vertical to the plate), but the aperture of these channels (<0.25 nm) does not allow usual molecules to diffuse in this direction. These characteristics make ferrierite a unique molecular sieve for catalysis, separation, ion exchange applications.

It is well known that the microporous nature of zeolite materials limits their sorption and occlusion ability and the accessibility to their internal surface to reactants or sorbates with sizes equivalent, or smaller, than the size of the micropores. To circumvent this limitation, secondary pore systems in the mesopore range size (2-50 nm) are necessary to increase their intracrystalline volume and improve transport and accessibility to the active surface (Chem. Commun., 2010, 46, 7840-7842).

Over the years many innovative post-synthesis routes, such as dealumination (Catal. Rev. Sci. Eng., 2003, 45, 297-319) and desilication (J. Phys. Chem. B, 2004, 108, 13062-13065), also named as ‘destructive’ approaches (Chem. Commun., 2010, 46, 7840-7842; Chem Cat Chem, 2011, 3, 67-81), have been developed for generating mesopores in zeolites. By 3D-TEM observations (electron tomography), it has been demonstrated (J. Phys. Chem. B, 2004, 108, 13062-13065) that most of the mesopores created upon dealumination by steaming followed by acid leaching of NH₄Y zeolite were randomly distributed cavities, with broad distributions of sizes, interconnected by channels and open to the external surface of the zeolite crystal. This pore architecture is commonly found in dealuminated zeolites. Framework desilication by alkaline attack of zeolite crystals is another effective method to generate mesopores in zeolites (J. Phys. Chem. B, 2004, 108, 13062-13065). The mesoporous network thus formed is similar to the one produced by dealumination and is open to the external surface and easily accessible from mercury intrusion porosimetry data (Part. Part. Syst. Charact., 2006, 23, 101-106). The alkaline desilication method has been successfully applied to treat many silica rich zeolites, such as MFI (J. Phys. Chem. B, 2004, 108, 13062-13065), MOR (J. Catal., 2007, 251, 21-27), FAU (Angew. Chem., Int. Ed., 2010, 49, 10074-10078), FER (J. Catal., 2009, 265, 170-180), etc.

As an alternative to better control the size and the distribution of mesopores in zeolite crystals, “constructive methods” have been introduced more recently, eventually in combination with destructive ones (Chem. Commun., 2010, 46, 7840-7842). A constructive method, also called “zeolite recrystallization” involves first the controlled partial dissolution of the zeolite in alkaline solution and then its re-assembly in the presence of surfactants (i.e. CTAB) through hydrothermal treatment. This method was proposed for the preparation of mesoporous MOR, MFI, BEA, FER, etc., and allows introducing ordered mesopores (MCM-41-type) into zeolite crystals (Pure Appl. Chem., 2004, 76, 1647-1658; Microporous Mesoporous Mater., 2011, 146, 201-207; Appl. Catal. A, 2012, 441-442, 124-135; Chem. Soc. Rev., 42, 3671-3688). The resulting materials generally consist of composite mixtures of mesoporous disordered and ordered portions of the original zeolite.

Until now most of the reported desilication and recrystallization procedures are performed in solutions of strong inorganic or organic bases, including NaOH, TMAOH, etc.

Because all T-sites of ferrierite are bonded to five rings, the framework is very stable and harsh conditions, such as the use of NaOH concentrations of 0.5 mol/L, temperatures of 80° C. and treatment times of 3 h, were required to extract silicon, leading to mesoporosity in H-FER zeolite with Si/Al ratio of 27. Hierarchical micro/mesoporous FER zeolite was also successfully synthesized by dissolution of H-FER (Si/Al=27) first in NaOH solution and then hydrothermal recrystallization in the presence of cethyltrimethylammonium bromide (CTAB). These NaOH desilicated materials led to improved catalytic performance in skeletal isomerization of 1-butene, but with no possibility to control the extent of mesopore creation as well as the pore size and the distribution of the mesopores.

Aqueous sodium carbonate (Na₂CO₃) solution, also named soda, one of the widely-used mild base salts in industry, was also reported to induce partial selective dissolution of framework silicon in the interior of ZSM-5 zeolite crystals (Zeolite, 1992, 12, 776-779). Recently, ZSM-5 microboxes composed of a thin shell and large hollow core were synthesized by a mild alkaline treatment of ZSM-5 single-crystals in Na₂CO₃ solution (J. Catal., 2008, 258, 243-249; J. Mater. Chem., 2008, 18, 3496-3500), which can increase the propylene selectivity in methanol-to-propylene reaction and the catalysis activity of cumene cracking and α-pinene isomerization.

Ogura et al in Applied Catalysis A vol 219 no 1-2, 2001 pages 33-43 disclose the alkali treatment of ZSM5 under atmospheric pressure at a maximum temperature of 80° C. The ZSM-5 crystals obtained according to the method described by Ogura present cracks and faults on their surface depending on the operating conditions.

In WO 2012/084276 relates to the desilication of USY zeolite (a 3D zeolite) via alkaline treatment under atmospheric pressure.

Dessau et al. in Zeolites vol 12 no 7 1992 pages 776-779 disclose describe the partial dissolution of the interior of ZSM-5 crystals with the treatment under reflux and under atmospheric pressure of the crystallites with aqueous solution of 0.5M of Na₂CO₃. Hollowed crystals of ZSM-5 are obtained via this process.

Groen et al. in Microporous and Mesoporous Materials vol 69 no 1-2, 2004 pages 29-34 disclose the production of intracrystalline mesoporosity in zeolites via desilication in alkaline medium. Desilication is performed at a maximum temperature of 85° C. under atmospheric pressure and leads to partial dissolution of the zeolite crystals.

Bonilla et al. in Journal of Catalysis, vol 265 no 2, 2009, pages 170-180 relate to the desilication of ferrierite with alkali treatment under atmospheric pressure and at a maximum temperature of 90° C. The ferrierite obtained do not have a defined mesopore size but instead have a broad distribution of large mesopores.

In US 2008/138274 relates to mesoporisation of USY, a 3D zeolite, with an alkaline solution.

Aguirre et al. in Revista de al Sociedad Quimica del Peru, 74(4), 291-297, 2008 disclose the preparation of zeolite MOR/MCM41 via hydrothermal treatment.

In US 2005/239634 relates to mesoporisation of MCM41, a 3D zeolite, with an alkaline solution.

Though Na₂CO₃ solution has been reported for the desilication to ZSM-5 at ambient temperature and pressure, until now the method has not been claimed for the preparation of other mesoporous zeolites.

### DESCRIPTION OF THE INVENTION

We disclose here a new desilication route for crystalline zeolites with mono-dimensional (1D) and bi-dimensional (2D) microporosity performed under mild hydrothermal conditions and in a basic aqueous medium, using preferably an alkaline metal carbonate solution.

In one embodiment, the invention relates to a mesoporous zeolitic material being preferably FER possessing an ordered mono-dimensional (1D) or two-dimensional (2D) network of micropores (i.e. pores<2 nm in diameter) containing mesopores (i.e. pores with diameters in the range 2-50 nm) connected to the microporores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores. Such mesoporous zeolitic can preferably be characterized by their adsorption isotherm of the type IV or V according to the IUPAC classification being preferably measured using N2 BET adsorption method. The orientation of the mesopores is preferably determined via TEM.

Ordered mono-dimensional (1D) micropore architecture or two-dimensional (2D) interconnecting micropore architecture networks refer to the channel system of the mesoporous zeolitic material. It is known in the art and can be found for instance on the Zeolite database structures (see for instance www.iza-structure.org/databases/).

The composition can preferably be determined by elemental analysis using the EDX method. Crystal structure can preferably be analyzed by X-Ray diffraction with Bragg-Brentano geometry. Pore volumes can be calculated from the analysis of the sorption-desorption isotherms for nitrogen recorded at 77 K. The total pore volume (including micropore volume, volume of intracrystalline mesopores and volume of intercrystalline mespores) can be calculated from the total amount adsorbed at a relative pressure p/p0 of 0.95. The volume of micropores plus intracrystalline mesopores can be calculated using the as plots method applied to the desorption branch of the isotherm at p/p0=0.5. The micropore volume can be calculated using the as plots method applied to the fraction of the isotherm below p/p0=0.3. The distribution, the size and the orientation of the intracrystalline mesopores can be determined by Transmission Electron Microscopy (TEM).

Moreover, different from the previous art where H-type ferrierite with a Si/Al ratio of 27 (obtained by previous dealumination) is usually reported to be used as the starting material due to the easy extraction of silicon atoms from the framework, we report here the selective desilication of a as-synthesized low-silica Na/K-type ferrierite (NaKFER) with a Si/Al ratio of 9.2.

Finally, for the first time, we report a resulting material exhibiting a unique distribution of oriented 3D short cylinder-like mesopores or tablet-like mesopores, due to the selective dissolution of the crystal along both the 8-([010]) and 10-MR ([001]) channel directions, with a narrow and tunable distribution of sizes.

The term “zeolite” as used herein refers to both natural and synthetic microporous crystalline silicate silicate materials having a definite crystalline structure as determined by X-ray diffraction. Crystalline silicates (also called zeolites) are microporous crystalline inorganic polymers based on a framework of XO4 tetrahedra linked to each other by sharing of oxygen ions, where X may be trivalent (e.g. Al, B, . . . ) or tetravalent (e.g. Ge, Si, . . . ). A zeolite comprises a system of channels which may be interconnected with other channel systems or cavities such as side-pockets or cages. The channel systems may be three-dimensional, two-dimensional or one-dimensional.

The term “intracrystalline mesopore” as used herein corresponds to mesopores which are located within a zeolite crystal.

The term “intercrystalline mesopore” as used herein corresponds to mesopores which are located between zeolite crystals.

The pore volume and the pore diameter are preferably measured via isotherm adsorption method (BET) for instance according to ASTM D4365.

The type of zeolite suitable for use in the process as the parent zeolite can be selected from the group consisting of:
mono-dimensional (1D) micropore architecture.
or two-dimensional (2D) interconnecting micropore architecture.

In a preferred embodiment, suitable parent zeolites for use in the process having mono-dimensional (1D) micropore architecture comprise a topology selected from the groups MTT (ZSM-23), TON (ZSM-22, Theta-1, NU-10), EUO (ZSM-50, EU-1), MOR.

In a preferred embodiment, suitable parent zeolites for use in the process having two-dimensional (2D) interconnecting micropore architecture comprise a topology selected from the groups FER (ferrierite, FU-9, ZSM-35), MWW (MCM-22, PSH-3, ITQ-1, MCM-49), MFS (ZSM-57), ZSM-48. The FER group is the most preferred group.

In a preferred embodiment, suitable parent materials are zeolites not subjected to modification treatments such as, and without being limited to, dealumination, steaming, acid leaching, desilication treatments.

The parent crystalline silicate is such that the Si/Al ratio ranges more advantageously from 5 to 100, preferably from 9 to 90.

In another embodiment, the invention relates to a process for preparing a mesoporous zeolitic material possessing a mono-dimensional or bi-dimensional channel system and being preferably FER, comprising the following steps:
i) contacting a parent zeolitic material with a basic aqueous solution containing at least one weak base i.e. a base having a pKa of at least 7 preferably at least 9 to at most 14 in water, preferably an alkaline metal carbonate, at a concentration ranging from 0.5M to 3M, preferably between 1M to 2M, more preferably from 1.25M to 2M to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., preferably from 120 to 150° C., more preferably from 130° C. to 150° C., under a pressure from 2 to 20 bara, preferably between 2 and 15 bara said pressure being preferably autogeneously generated,
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
vii) calcining the zeolite obtained at step (vi), and recovering the mesoporous zeolitic material
wherein optionally the ratio of said parent zeolitic material to said basic aqueous solution in said first composition ranges from 0.02 to 0.05 g/mL, preferably 0.03 to 0.04 g/mL and is most preferably of 0.0334 g/mL.

In another embodiment, the invention relates to a process for preparing the mesoporous zeolitic material possessing a mono-dimensional or bi-dimensional channel system and being preferably FER, comprising the following steps:
i) contacting a parent zeolitic material with a basic aqueous solution containing at least a strong base i.e. a base that is totally dissociated in water or a base having a pKa higher than 14 such as an alkaline hydroxide base at a concentration ranging from 0.2M to 0.3M, more preferably at 0.25M, to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., preferably from 120 to 140° C., more preferably at 130° C., under a pressure from 2 to 20 bara, preferably between 2 and 15 bara said pressure being preferably autogenously generated,
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
vii) calcining the zeolite obtained at step (vi), and recovering the mesoporous zeolitic material
wherein optionally the ratio of said parent zeolitic material to said basic aqueous solution in said first composition ranges from 0.02 to 0.05 g/mL, preferably 0.03 to 0.04 g/mL and is most preferably of 0.0334 g/mL.

A process for preparing the mesoporous zeolitic material possessing a mono-dimensional or bi-dimensional channel system and being preferably FER, comprising the following steps:
i) contacting a parent zeolitic material with a basic aqueous solution containing at least one weak base (in particular an alkaline metal carbonate) i.e. a base having a pKa ranging from 7 to 13.5 at a concentration ranging from 1M to 2M, and/or one strong base i.e. a base that is totally dissociated in water such as an alkaline hydroxide base at a concentration ranging from 0.2M to 0.5M in presence of a mesopore organic structure directing agent, to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., preferably from 120 to 150° C., more preferably from 130° C. to 150° C., under a pressure from 2 to 20 bara, preferably between 2 and 15 bara, said pressure being preferably autogenously generated
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
vii) calcining the zeolite obtained, at step (vi) and recovering the mesoporous zeolitic material
   wherein optionally the ratio of said parent zeolitic material to said basic aqueous solution in said first composition ranges from 0.02 to 0.05 g/mL, preferably 0.03 to 0.04 g/mL and is most preferably of 0.0334 g/mL.

In a most preferred embodiment, said mesoporous zeolitic material prepared according to any of the above process possesses an ordered mono-dimensional (1D) or two-dimensional (2D) network of micropores (i.e. pores<2 nm in diameter) containing mesopores (i.e. pores with diameters in the range 2-50 nm) connected to the microporores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores.

In a most preferred embodiment, said mesoporous zeolitic material prepared according to any of the above process possesses, has a network of micropores has a geometry consistent with one of MTT (ZSM-23), TON (ZSM-22, Theta-1, NU-10), EUO (ZSM-50, EU-1), FER (ferrierite, FU-9, ZSM-35), MWW (MCM-22, PSH-3, ITQ-1, MCM-49), MFS (ZSM-57), and ZSM-48.

Autogeneous pressure refers to a pressure at least higher than the atmospheric pressure and self generated by the heating. Autogeneous pressure is generally obtained via heating of a closed vessel.

The gist of the invention lies in a particularly versatile desilication process of zeolite leading to mesoporous materials with unique properties. Either a strong or a weak base can be used in the processes described above. The processes described above are particularly suitable for zeolite such as FER. Indeed FER are known to be difficult to desilicate: depending on the operating condition FER is either not desilicated at all or fully dissolved. The processes described above allow a controlled desilication of zeolite and in particular of FER.

In one embodiment, the base used for the desilication route according to the invention is a strong base and/or a weak base. Preferably the base is a weak base having a pKa above 9, more preferably above 10, even more preferably chosen among an alkaline metal carbonate, such as sodium, potassium, lithium ammonium carbonate. In a more preferred embodiment, the weak base used in the disclosed invention is sodium carbonate i.e. Na₂CO₃. The alkaline metal carbonate is preferably chosen among Na₂CO₃, (NH4)2CO₃, NaHCO3 or K2CO3 or any mixture thereof.

In one embodiment, heating of the composition is done at a temperature from 101 to 150° C., preferably from 120 to 150° C., more preferably from 130° C. to 150° C., under optionally autogenous pressure from 1 preferably 2 to 20 bara, preferably between 1 preferably 2 and 15 bara.

The unit “bara” refers to “bar absolute”. Measurement of the pressure can be “absolute” or “relative”. Relative pressure is made by comparison with the atmospheric pressure. It is the measure made by most nanometers; when the nanometer indicates zero the pressure is equal to the atmospheric pressure. On the other hand, the absolute pressure is the pressure usually used in thermodynamic. The difference between the relative and the absolute pressure is the atmospheric pressure (1 bar).

Alternatively the base is a strong base, preferably an alkaline hydroxide, alkaline earth hydroxide, tetraalkylammonium hydroxide; more preferably sodium hydroxide. In one embodiment, the organic structure directing agent is typically a surfactant, which is solid under ambient temperature and pressure conditions. Suitable surfactant that can be employed include cationic, ionic, neutral surfactants and/or combinations of these. Exemplary surfactants include for example, hexadecyltrimethylammonium bromide, or cetyltrimethylammonium bromide (CTAB). Another type of suitable surfactant includes recyclable surfactants, characterized in that they are able to generate a micellization upon the effect of the variation of a physico-chemical parameter (pH, temperature, ionic strength). A non limiting example of mesopore structure directing agent is an oligomeric or polymeric chain bearing at least one ionic function and rendered amphiphilic upon the effect of the variation of a physico-chemical parameter, preferably chosen among pH, temperature and ionic strength and is preferably selected among:
a statistical copolymer of ethylene and propylene functionalized by a quaternary ammonium salt, such as Jeffamines, the molecular size of which varying from 140 to 5000 g/mol and the ethylene oxide/propylene oxide molar ratio of which varying from 0.01 to 5, more preferably between 0.1 to 1, most preferably between 0.1 to 0.5, said Jeffamines being quaternized on their primary amine wherein the amino group of the mesopore-templating agent is preferably quaternized, most preferably with chloride or bromide or hydroxide; or

is preferably a Jeffamine selected among Jeffamine M600 and Jeffamine M2005 wherein the amino group of the mesopore-templating agent is preferably quaternized, most preferably with chloride or bromide or hydroxide. None limited examples of recyclable surfactants can be found in WO2016005277 which is thereby incorporated by reference.

Examples of recyclable surfactants include for example commercially available Jeffamines, which can be quaternized or not.

The final material obtained according to the present invention can be subjected to various treatments before use in catalysis including, ion exchange, modification with metals (in a not restrictive manner alkali, alkali-earth, transition, rare earth elements or noble metals), external surface passivation, modification with P-compounds, steaming, acid treatment or other dealumination methods, or combination thereof.

In another first embodiment, the invention can be described as a mesoporous zeolitic material possessing an ordered mono-dimensional (1D) or two-dimensional (2D) network of micropores (ie pores<2 nm in diameter) containing mesopores (pores with diameters in the range 2-50 nm) connected to the microporores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores.

In a second embodiment, the invention relates to a mesoporous zeolitic material according to the embodiment 1, which network of micropores has a geometry consistent with one of MTT (ZSM-23), TON (ZSM-22, Theta-1, NU-10), EUO (ZSM-50, EU-1), FER (ferrierite, FU-9, ZSM-35), MWW (MCM-22, PSH-3, ITQ-1, MCM-49), MFS (ZSM-57), and ZSM-48.

In embodiment 3, the invention relates to a process for preparing the mesoporous zeolitic material of embodiments 1 or 2, comprising the following steps:
i) contacting a parent zeolitic material with a basic aqueous solution containing at least one weak base, preferably an alkaline metal carbonate, at a concentration ranging from 0.5M to 3M, preferably between 1M to 2M, more preferably from 1.25M to 2M to obtain a first composition,
ii) heating said first composition at a temperature from 101 to 150° C., preferably from 120 to 150° C., more preferably from 130° C. to 150° C., under optionally autogeneous pressure from 1 to 20 bara, preferably between 1 and 15 bara,
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
vii) calcining the zeolite obtained at step (vi), and recovering the mesoporous zeolitic material.

In embodiment 4, the invention relates to a process for preparing the mesoporous zeolitic material of embodiments 1 or 2, comprising the following steps:
i) contacting a parent zeolitic material with a basic aqueous solution containing at least a strong base such as an alkaline hydroxide base at a concentration ranging from 0.2M to 0.3M, more preferably at 0.25M, to obtain a first composition,
ii) heating said first composition at a temperature from 100 to 150° C., preferably from 120 to 140° C., more preferably at 130° C., under optionally autogeneous pressure from 1 to 20 bara, preferably between 1 and 15 bara,
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
vii) calcining the zeolite obtained at step (vi), and recovering the mesoporous zeolitic material.

In embodiment 5, the invention relates to a process for preparing the mesoporous zeolitic material of embodiments 1 or 2, comprising the following steps:
i) contacting a parent zeolitic material with a basic aqueous solution containing at least one weak base (in particular an alkaline metal carbonate) at a concentration ranging from 1M to 2M, and/or one strong base such as an alkaline hydroxide base at a concentration ranging from 0.2M to 0.5M in presence of a mesopore organic structure directing agent, to obtain a first composition,
ii) heating said first composition at a temperature from 100 to 150° C., preferably from 120 to 150° C., more preferably from 130° C. to 150° C., under optionally autogenous pressure from 1 to 20 bara, preferably between 1 and 15 bara,
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with preferably distilled water, preferably to neutral pH,
   calcining the zeolite obtained, at step (vi) and recovering the mesoporous zeolitic material.

In embodiment 6, the invention relates to a process according to embodiment 5, wherein the mesopore structure directing agent is a surfactant, preferably cetyltrimethylammonium bromide (CTAB).

In embodiment 7, the invention relates to a process according to embodiment 5, wherein the mesopore structure directing agent is a recyclable surfactant able to generate a micellization upon the effect of the variation of a physico-chemical parameter (pH, temperature, ionic strength).

In embodiment 8, the invention relates to a process according to embodiment 5, in wherein the mesopore structure directing agent is selected among optionally quaternized Jeffamines.

In embodiment 9, the invention relates to a process according to one any of embodiments 3 and 5-8, wherein the alkaline metal in the alkaline metal carbonate is selected among ammonium, sodium and potassium, and/or their mixtures.

In embodiment 10, the invention relates to a process according to one any of embodiments 4-8, wherein the alkaline hydroxide is selected among ammonium, sodium and potassium hydroxides, and their mixtures.

In embodiment 11, the invention relates to a process according to one any of embodiments 3-10, wherein the parent zeolitic material is a mono-dimensional (1D) micropore architecture zeolite selected from the groups MTT (ZSM-23), TON (ZSM-22, Theta-1, NU-10), EUO (ZSM-50, EU-1).

In embodiment 12, the invention relates to a process according to one any of embodiments 3-10, wherein the parent zeolitic material is two-dimensional (2D) inter-connecting micropore architecture zeolite selected from the groups FER (ferrierite, FU-9, ZSM-35), MWW (MCM-22, PSH-3, ITQ-1, MCM-49), MFS (ZSM-57), ZSM-48.

In embodiment 13, the invention relates to a process according to embodiments 12, wherein the parent zeolitic material belongs to the FER group.

In embodiment 14, the invention relates to the use of an optionally formulated material obtained according to one any of embodiments 3-13, as refining or petrochemical catalyst.

In embodiment 15, the invention relates to the use of a material according to embodiment 1 or 2 as refining or petrochemical catalyst.

### EXPERIMENTALS

### Methods of Characterization

The composition of the samples has been determined by elemental analysis using the EDX method. EDX method is a global method allowing the titration of all elements form ppm level. Crystal structure was analyzed by X-Ray diffraction on a Bruker Lynx Eye diffractometer with Bragg-Brentano geometry and CuKα radiation (λ=0.15406 nm) as incident beam. Data were recorded by continuous scanning in the range 4-50°/2θ for studying the crystalline zeolite structure and in the range of 0.5-6°/2θ for the mesoporous structure, with an angular step size of 0.0197°/2θ and a counting time of 0.2 second per step. Pore volumes were calculated from the analysis of the sorption-desorption isotherms for nitrogen recorded at 77 K using a Micromeritics TriStar 3000. Prior to the isotherms acquisition, the samples were degassed under vacuum at 250° C. for 7 h. The total pore volume (including micropore volume, volume of intracrystalline mesopores and volume of intercrystalline mespores) was calculated from the total amount adsorbed at a relative pressure p/p0 of 0.95. The volume of micropores plus intracrystalline mesopores was calculated using the as plots method applied to the desorption branch of the isotherm at p/p0=0.5. The micropore volume was calculated using the as plots method applied to the fraction of the isotherm below p/p0=0.3.

The distribution, the size and the orientation of the intracrystalline mesopores was determined by Transmission Electron Microscopy (TEM) equipped with microdiffraction patterning using a Jeol 1200 electron microscope.

### Examples

Starting Materials

The following samples of ferrierite have been used as starting materials
FER1: NaKFER, with Si/Al of 9.2 was supplied by Tosoh Corporation under the code HSZ-720 KOA
FER2: HFER prepared by ion exchange of FER1 by NH4NO3 solution, followed by drying at 110° C. and calcination at 550° C. under air.

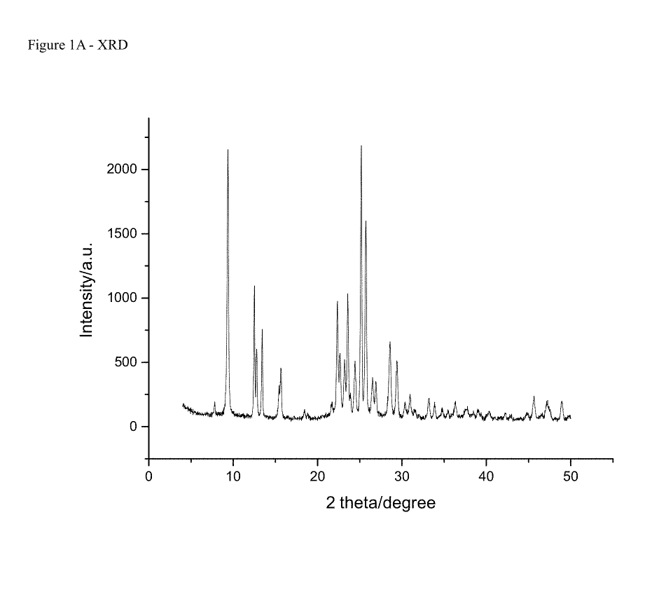

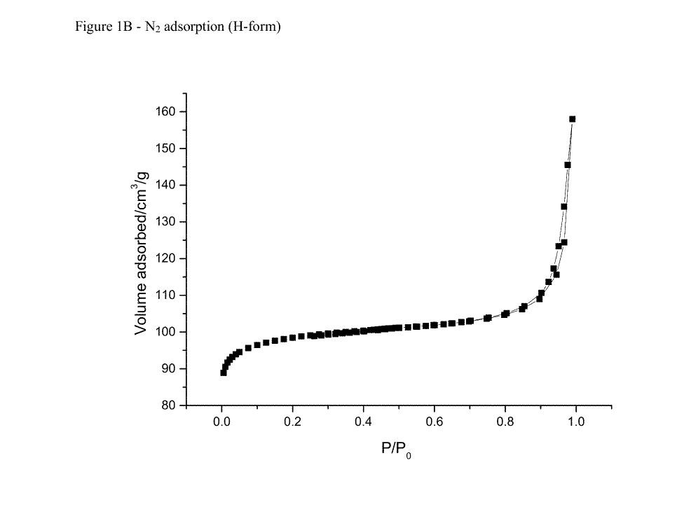

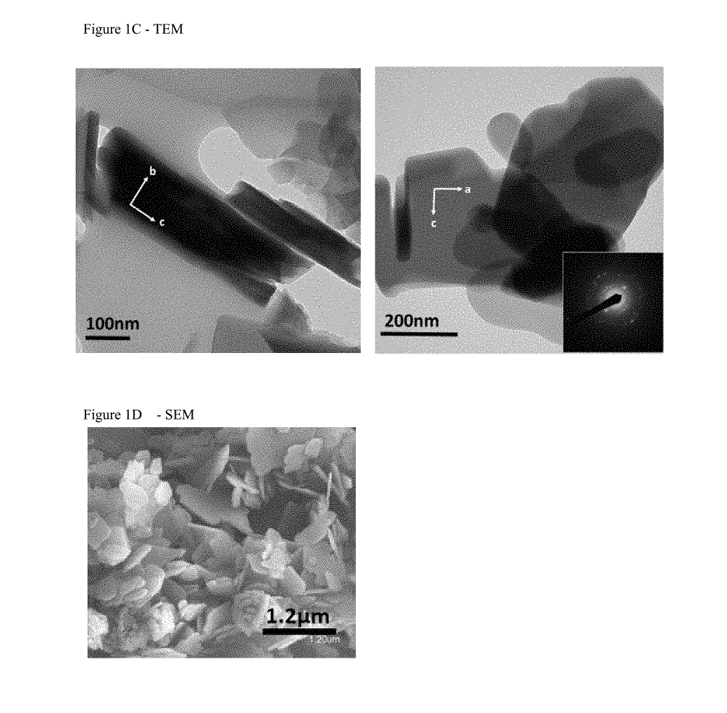

The XRD spectrum of FER1 is shown in FIG. 1A which shows the high cristallinity of the sample. Nitrogen sorption measurements performed on FER2 (FIG. 1B) reveal a type I isotherm with a high adsorption in micropores at low relative (p/p°) pressures. At relative pressures higher than 0.9 the amount adsorbed increases due to the condensation of nitrogen between the particles (interparticle mesopores). The sorption measurements are therefore characteristic of a microporous material which does not contain intracrystalline mesopores. TEM images of the crystals in the (010) and (100) directions and micro-diffractograms (FIG. 1C) confirm that the material is highly crystalline and free of intracrystalline mesopores. The composition and textural features of FER1 and FER 2 are given in Table 1.

The XRD, sorption isotherms and TEM images of samples FER1 are given in FIGS. 1 (A, B, C, D).

Abbreviations used in the examples, Micro=Micropores; Inter. Meso=Intercrystalline Mesopores, i.e. mesopores located between zeolite crystals; Intra. Meso=Intracrystalline Mesopores, i.e. mesopores located within a zeolite crystal.

TABLE 1
Composition and textural features of FER1 and FER2

| | Yield/ | | | | (Na + K)/ |
|---|---|---|---|---|---|
| Sample | % | Si/Al | Na/Al | K/Al | Al |
| FER1 | \ | 9.2 | 0.22 | 0.70 | 0.92 |

| | | Pore volume (cm³/g) | | | |
|---|---|---|---|---|---|
| | BET surface area | | | Inter. | Infra. |
| Sample | (m²/g) | Total | Micro | Meso | Meso |
| FER2 | 399 | 0.18 | 0.15 | 0.03 | 0 |

### Example 1 [According to the Invention]: Illustrates the Material Obtained and Mode of its Preparing

In a typical synthesis, 1.67 g of commercial NaKFER (FER1, Si/Al=9.2) was first mixed with 50 mL of 1.25 mol/L Na₂CO₃ solution (solid/solution=0.0334 g/mL), and then stirred for 30 min. The suspension was hydrothermally treated at 130° C. in a Teflon-lined stainless autoclave for 3 days. After cooling the autoclave to room temperature, the solid was filtered under vacuum and washed with de-ionized water repeatedly until pH=7. Finally the product was dried overnight at 80° C. to get the sample of DeFER1-1.25-130/3. Yields of the preparation were calculated from the ratio between the amount of solid recovered to the amount of parent solid engaged in the reaction.

The as-synthesized DeFER1-1.25-130/3 sample was ion-exchanged in 1.0 mol/L NH₄NO₃ solution for 6 h at room temperature, after dryness, the sample was calcined in air flow (100 mL/min) in a tubular furnace at 550° C. for 8 h, and the sample denoted as H-DeFER1-1.25-130/3 was obtained.

TABLE 2
Composition and textural features of
the material prepared in example 1

| Example 1 | Yield/% | Si/Al | Na/Al | K/Al | (Na + K)/Al |
|---|---|---|---|---|---|
| DeFER1- | 76.7 | 7.9 | 0.51 | 0.39 | 0.90 |
| 1.25-130/3 | | | | | |

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 1 | (m²/g) | tal | Micro | Meso | Meso | Micro | Micro |
| H-DeFER1- | 341 | 0.21 | 0.15 | 0.03 | 0.03 | 0.4 | 0.2 |
| 1.25-130/3 | | | | | | | |

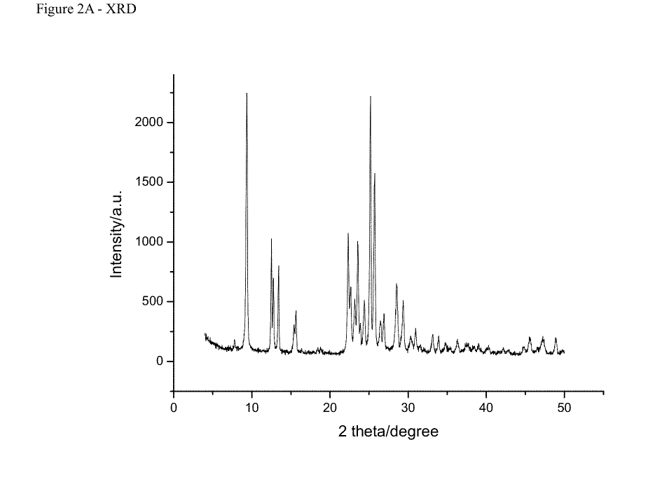

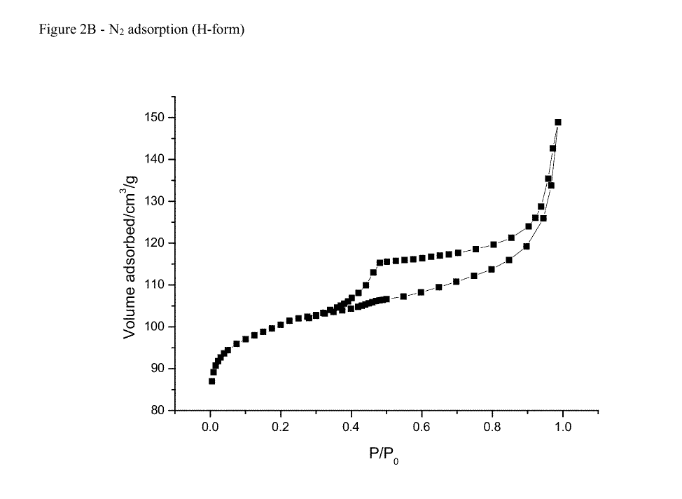

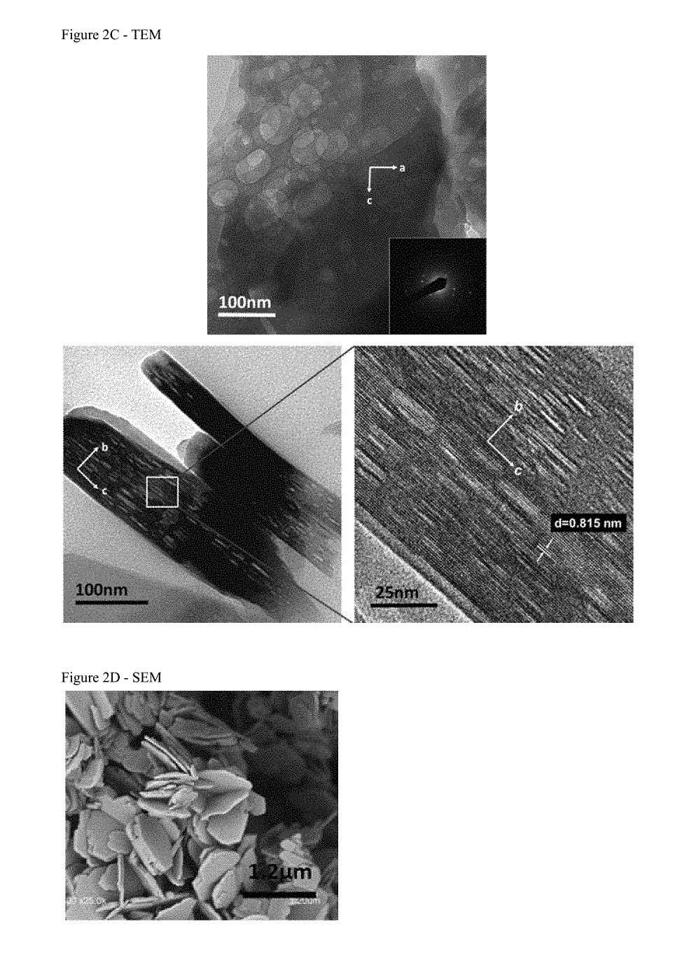

The XRD diffractogram of DeFER1-1.25-130/3 (FIG. 2A) shows that the crystallinity of the parent material has been preserved. The nitrogen isotherm (FIG. 2B, sample H-DeFER1-1.25-130/3) shows the appearance of a hysteresis loop with an abrupt closing branch around p/p° of 0.42 characteristic of a cavitation phenomenon associated with the presence of mesopores connected to the exterior of the crystal by restrictions smaller than ca. 3-4 nm. Compared to the parent material, the micropore volume has been barely modified and 0.03 mL/g of intracrystalline mesopores have been generated. TEM of the crystals in the (010) and (100) directions (FIG. 2C) shows that intracrystalline mesopores have been created throughout the whole crystal. The mesopores appear as clear zones in the micropgraphs. Seen in the (010) direction, they appear as quasi-circular with diameters in the range 20-70 nm. Examination of the crystals in the (100) direction shows that the mesopores consist of elongated voids of 2 to 5 nm in width, running parallel to the 10 MR channel of the microcrystalline structure. In a 3D representation, the mesopores created by the treatment in the Na₂CO₃ solution are described as flat elongated boxes, with a high aspect ratio (diameter to length, 10-30) oriented in the direction of the main channel of the ferrierite structure and connected to each other via the 10 MR of the framework.

### Examples 2 [According to the Invention] Showing that the Volume, and Aspect Ratio of the Intracrystalline Mesopores can be Tuned by Changing the Concentration of the Na₂CO₃ Solution

The same procedure as in example 1 has been applied by using Na₂CO₃ solutions with a different concentration of 0.5 mol/L. Thus 1.67 g of commercial NaKFER (FER1, Si/Al=9.2) was mixed with 50 mL of 0.5 mol/L Na₂CO₃ solution to yield DeFER1-0.5-130/3.

TABLE 3
Composition and textural features of
the material prepared in example 2

| Example 2 | Yield/% | Si/Al | Na/Al | K/Al | (Na + K)/Al |
|---|---|---|---|---|---|
| DeFER1-0.5- | 84.9 | 8.4 | 0.35 | 0.54 | 0.89 |
| 130/3 | | | | | |

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 2 | (m²/g) | tal | Micro | Meso | Meso | Micro | Micro |
| H-DeFER1- | 340 | 0.2 | 0.15 | 0.03 | 0.02 | 0.33 | 0.13 |
| 0.5-130/3 | | | | | | | |

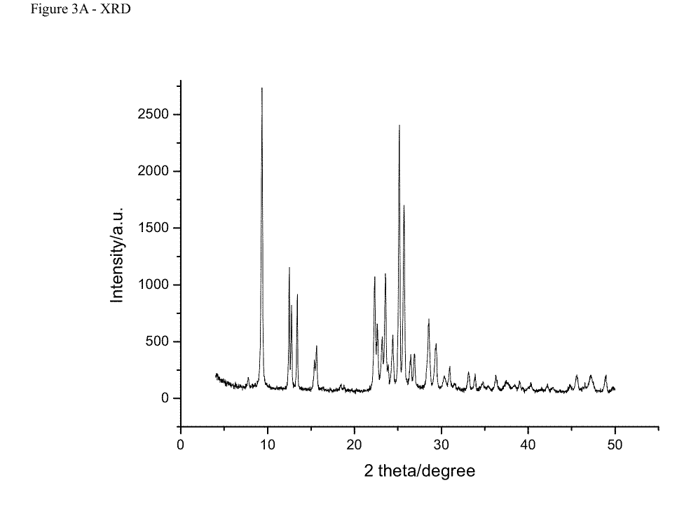

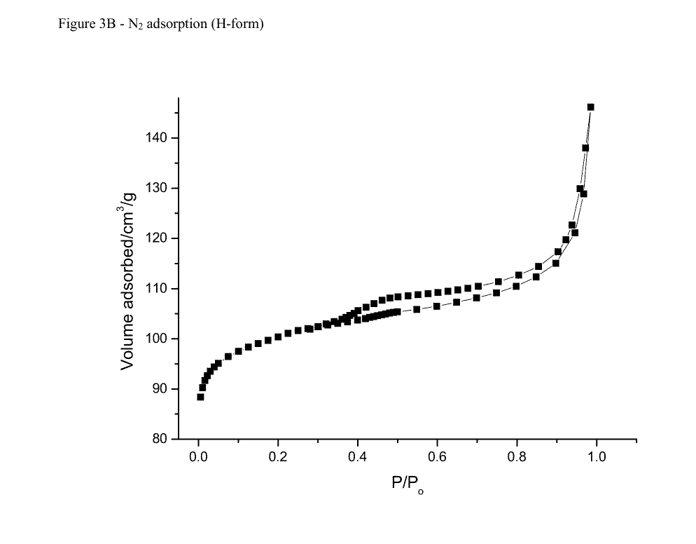

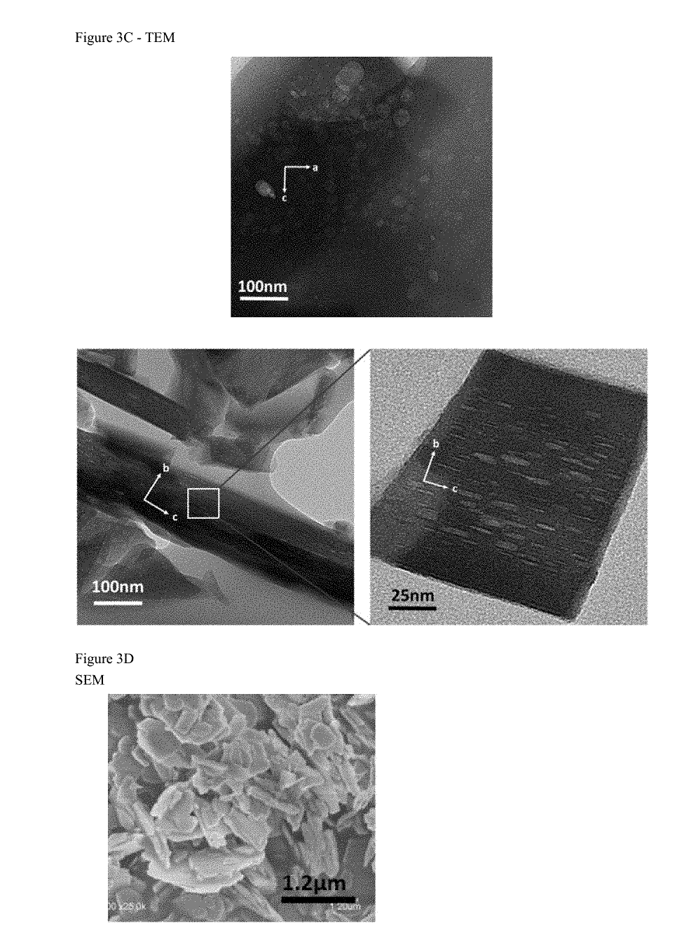

Decreasing the concentration of the Na₂CO₃ solution down to 0.5 mol/L leads to a crystalline material (FIG. 3A) with a lower amount of intracrystalline occluded mesopores (FIG. 3B, Table 3). The mesopores have an average diameter of 20 nm and a width in the range 1-5 nm (FIG. 3C). Their unique orientation is clearly visible in the TEM micrographs.

### Examples 3 [According to the Invention] Showing that the Volume, and Aspect Ratio of the Intracrystalline Mesopores can be Tuned by Changing the Concentration of the Na₂CO₃ Solution

The same procedure as in example 1 has been applied by using Na₂CO₃ solutions with a different concentration of 2 mol/L. Thus, 1.67 g of commercial NaKFER (FER1, Si/Al=9.2) was mixed with 50 mL of 2 mol/L Na₂CO₃ solution to yield DeFER1-2-130/3 (Example 3).

TABLE 4
Composition and textural features of
the material prepared in example 3

| Example 3 | Yield/% | Si/Al | Na/Al | K/Al | (Na + K)/Al |
|---|---|---|---|---|---|
| DeFER1-2- | 65.5 | 6.5 | 0.60 | 0.35 | 0.95 |
| 130/3 | | | | | |

| | BET | Pore volume/cm³/g | | | | | |
|---|---|---|---|---|---|---|---|
| | surface | | | | | | Intra. |
| | area/ | To- | | Inter. | Intra. | Meso/ | Meso/ |
| Example 3 | m²/g | tal | Micro | Meso | Meso | Micro | Micro |
| H-DeFER1- | 423 | 0.26 | 0.15 | 0.03 | 0.08 | 0.73 | 0.53 |
| 2-130/3 | | | | | | | |

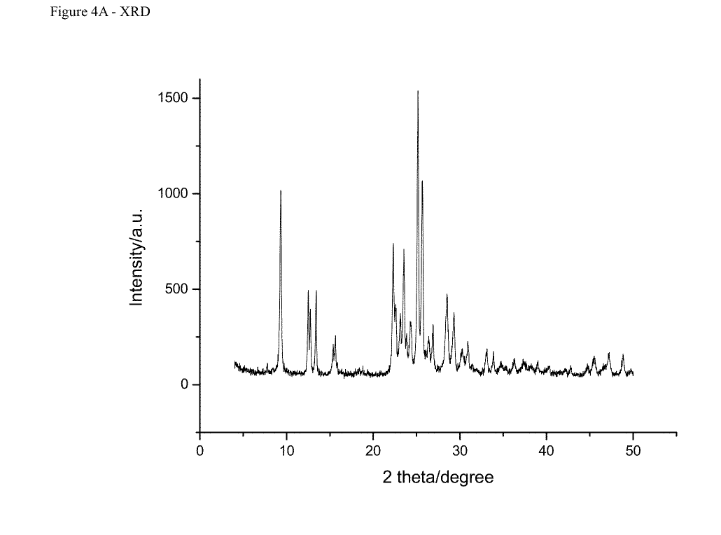

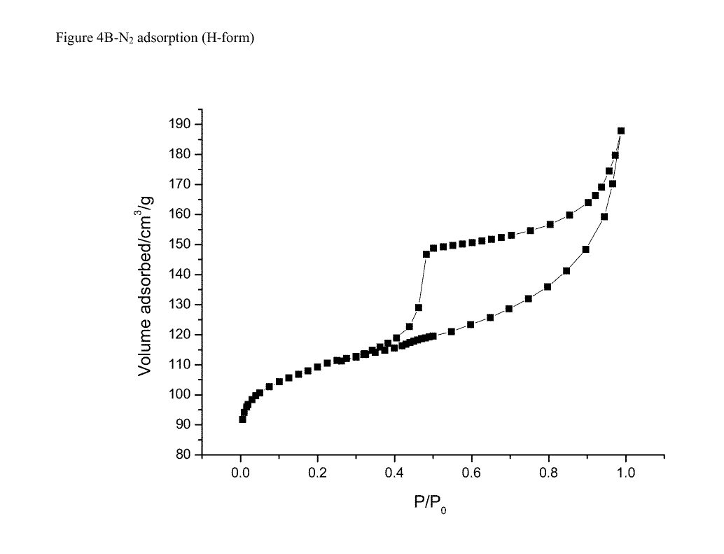

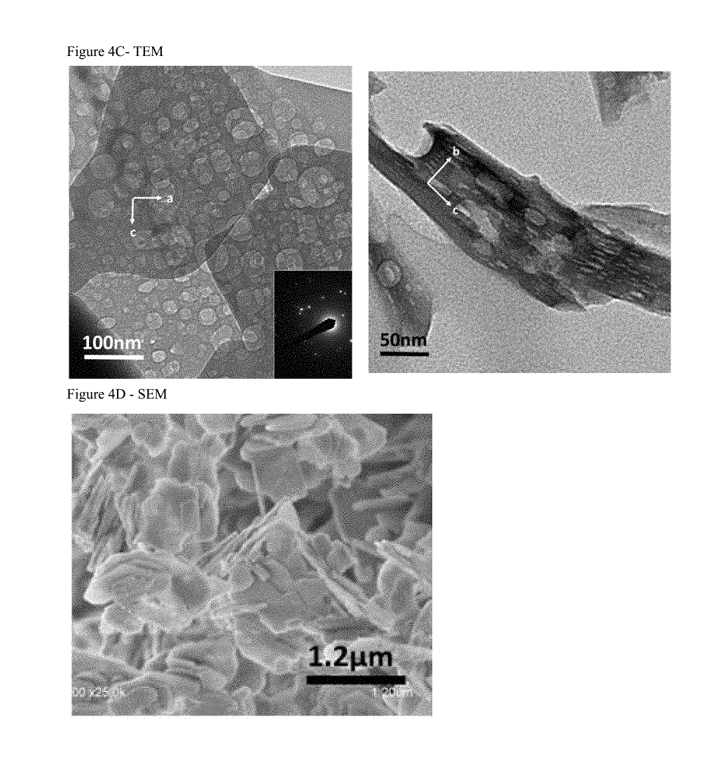

This example shows that by increasing the concentration of the Na₂CO₃ solution to 2 mol/L, a significant volume of intracystalline occluded mesopores can be created without loss of crystallinity (FIGS. 4A, 4B, Table 4). The mesopores have an average pore diameter in the (010) direction of 20-80 nm and a width of 5-20 nm in the (100) direction parallel to the pore channel of the zeolite (FIG. 4C).

### Example 4 [According to the Invention] Shows that HFER (FER2, Si/Al=9.2) can be Also Desilicated to Produce the Material we Claim

The same procedure used in example 1 has been applied to FER2 in order to produce DeHFER2-1.25-130/3.

TABLE 5
Composition and textural features
of material prepared in example 4

| Example 4 | Yield/% | Si/Al | Na/Al | K/Al | (Na + K)/Al |
|---|---|---|---|---|---|
| DeHFER2- | 74.9 | 7.2 | 0.67 | \ | \ |
| 1.25-130/3 | | | | | |

| | BET | Pore volume (cm³/g) | | | | | |
|---|---|---|---|---|---|---|---|
| | surface | | | | | | Intra. |
| | area | To- | | Inter. | Intra. | Meso/ | Meso/ |
| Example 4 | (m²/g) | tal | Micro | Meso | Meso | Micro | Micro |
| H- | 418 | 0.22 | 0.15 | 0.03 | 0.04 | 0.5 | 0.29 |
| DeHFER2- | | | | | | | |
| 1.25-130/3 | | | | | | | |

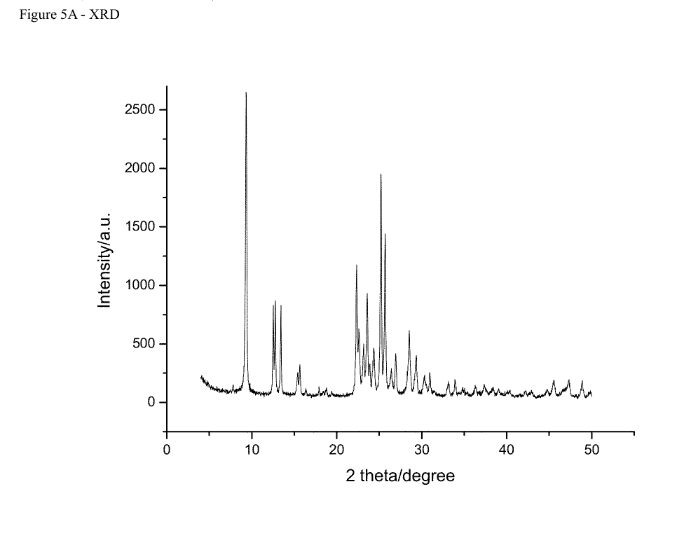

The procedure applied to a H form ferrierite allows to generate 0.04 mL/g of intracystalline occluded mesopores as shown by Table 5 and FIGS. 5A, 5B and 5C)

### Example 5 [Comparative Example] (DeFER1-1.25-80-3)

In the example, the same procedure as in example 1 has been applied by using Na₂CO₃ solutions with the concentration of 1.25 mol/L at 80° C. for 3 days. Thus 1.67 g of commercial NaKFER (FER1, Si/Al=9.2) was mixed with 50 mL of 1.25 mol/L Na₂CO₃ solution to yield DeFER1-1.25-80-3 (Example 5).

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 5 | (m²/g) | tal | Micro | Meso | Meso | Micro | Micro |
| H- | 410 | 0.19 | 0.15 | 0.03 | 0.01 | 0.27 | 0.07 |
| DeFER1- | | | | | | | |
| 1.25-80-3 | | | | | | | |

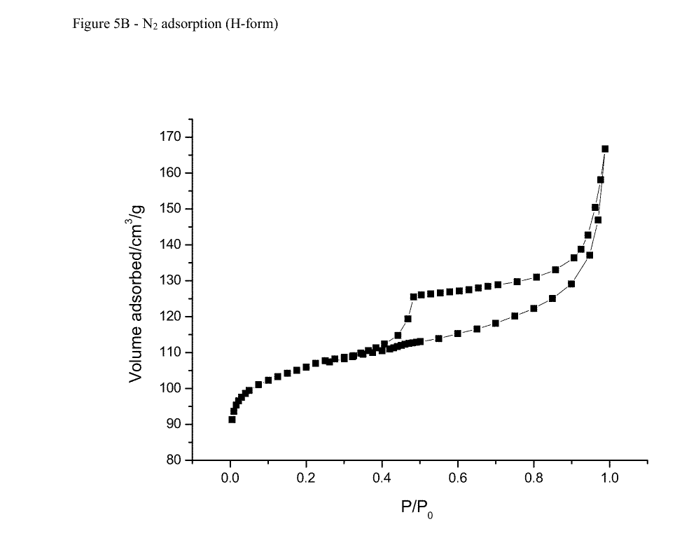

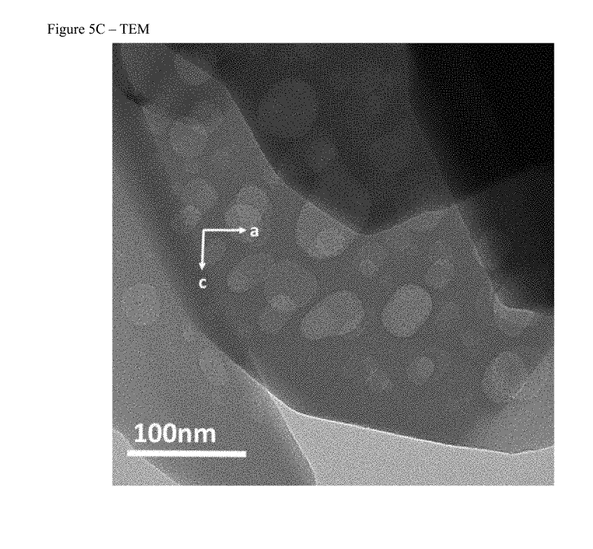

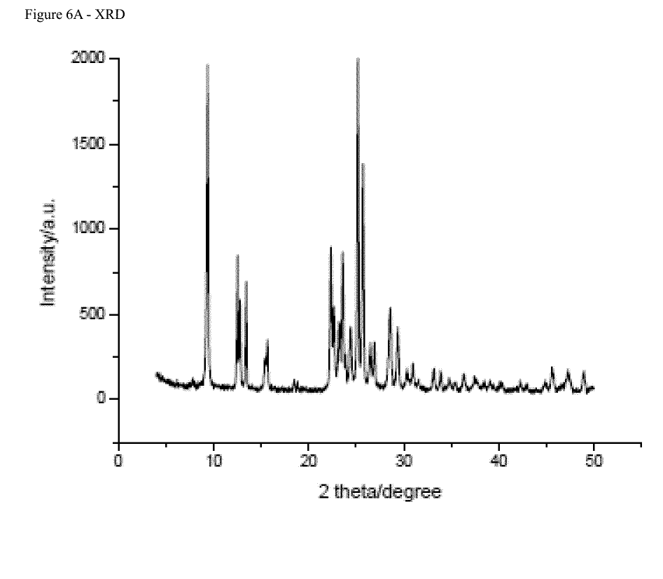

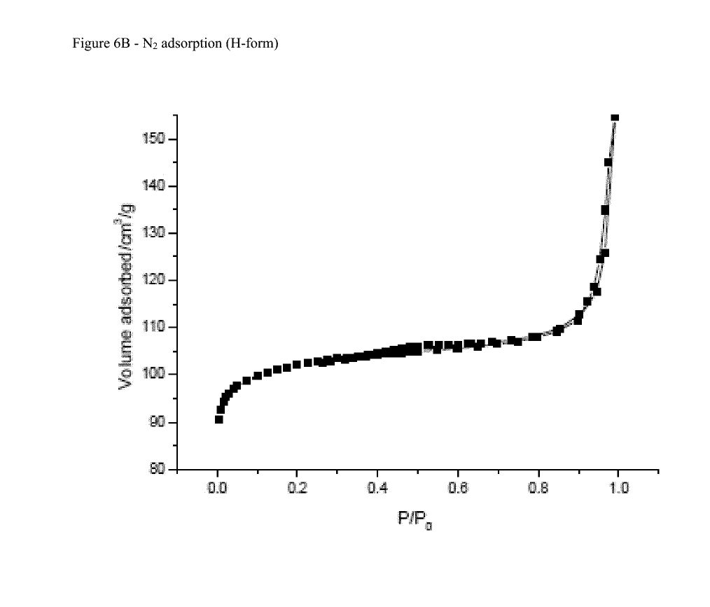

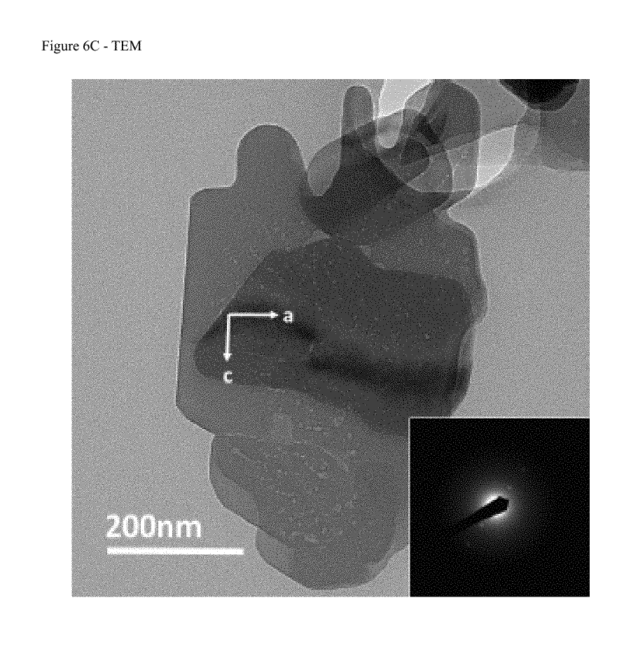

FIG. 6 show that by performing the reaction at too low temperature (80° C.), it is not possible to recover a mesoporous FER corresponding to the one we claim.

### Example 6 [According to the Invention] (DeFER1-1.25-150-3)

In the example, the same procedure as in example 1 has been applied by using Na₂CO₃ solutions with the concentration of 1.25 mol/L at 150° C. for 3 days. Thus 1.67 g of commercial NaKFER (FER1, Si/Al=9.2) was mixed with 50 mL of 1.25 mol/L Na₂CO₃ solution to yield DeFER1-1.25-150-3 (Example 6).

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 6 | (m²/g) | tal | Micro | Meso | Meso | Micro | Micro |
| H-DeFER1- | 342 | 0.21 | 0.14 | 0.03 | 0.04 | 0.5 | 0.29 |
| 1.25-150-3 | | | | | | | |

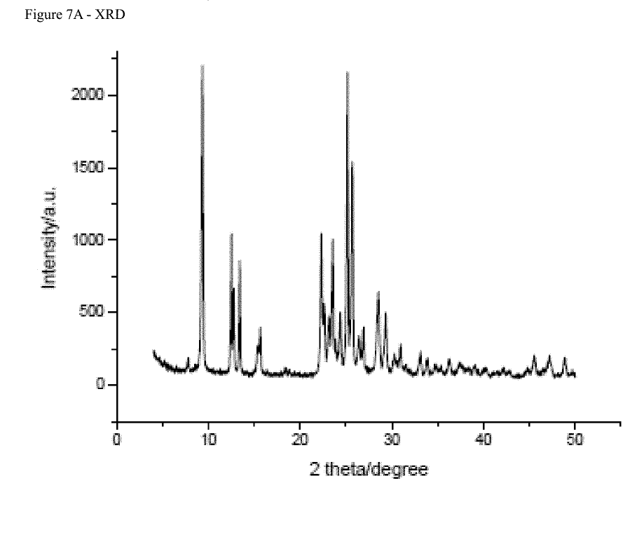

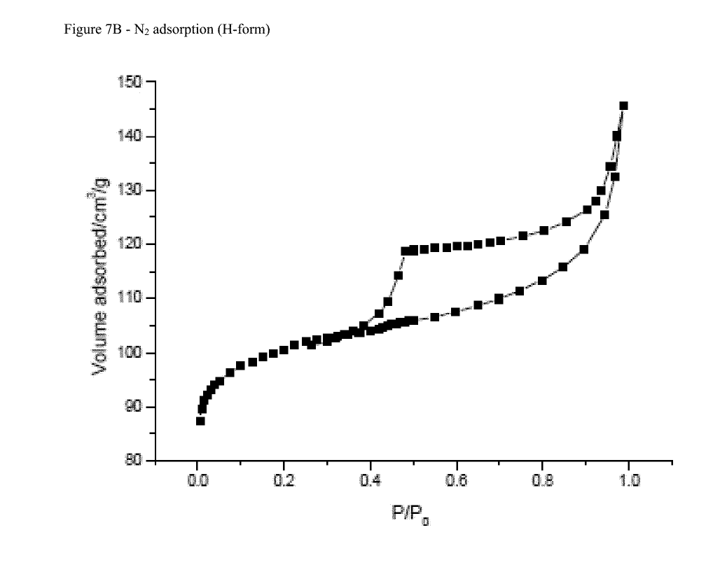

FIG. 7 show that the material obtained corresponds to the one we claim due to a proper reaction temperature.

### Example 7 [According to the Invention]: Effect of the Duration of the Hydrothermal Treatment

In the example, the same procedure as in example 1 has been applied by using Na₂CO₃ solutions with the concentration of 1.25 mol/L at 130° C. for different duration (6 hours, 24 hours, 72 hours). Thus 1.67 g of commercial NaKFER (FER1, Si/Al=9.2) was mixed with 50 mL of 1.25 mol/L Na₂CO₃ solution to yield DeFER1-1.25-130/x, x being the duration of the hydrothermal treatment in hours (Example 7).

| Example 7 | Yield/% | Si/Al | Na/Al | K/Al | (Na + K)/Al |
|---|---|---|---|---|---|
| FER1 | — | 9.2 | 0.22 | 0.7 | 0.92 |
| DeFER1- | 84.3 | 9.3 | 0.47 | 0.5 | 0.97 |
| 1.25-130/6 | | | | | |
| DeFER1- | 78 | 7.8 | 0.51 | 0.38 | 0.89 |
| 1.25-130/24 | | | | | |
| DeFER1- | 76.7 | 7.9 | 0.51 | 0.39 | 0.90 |
| 1.25-130/72 | | | | | |

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 7 | (m²/g) | tal | Micro | Meso | Meso | Micro | Micro |
| FER2 | 399 | 0.18 | 0.15 | 0.03 | 0 | 0.2 | 0 |
| H-DeFER1- | 347 | 0.2 | 0.15 | 0.03 | 0.02 | 0.33 | 0.13 |
| 1.25-130/6 | | | | | | | |
| H-DeFER1- | 361 | 0.21 | 0.15 | 0.03 | 0.05 | 0.53 | 0.33 |
| 1.25-130/24 | | | | | | | |
| H-DeFER1- | 341 | 0.21 | 0.15 | 0.03 | 0.03 | 0.4 | 0.2 |
| 1.25-130/72 | | | | | | | |
| (ex 1) | | | | | | | |

Example 7 shows that even after 6 hours of hydrothermal treatment under the applied conditions, we obtain a mesoporous FER exhibiting occluded mesopores which are oriented in the same direction as the micropores of the FER starting material.

### Summary of Examples 1 to 7:

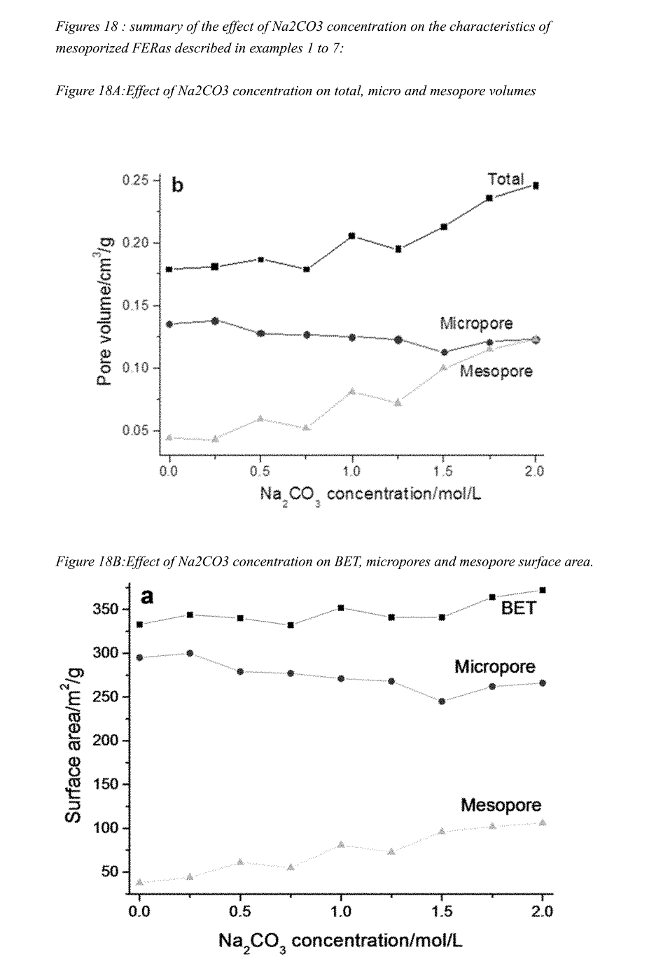

The examples 1 to 7 clearly show that a mesoporous FER exhibiting the following characteristics:
an ordered uni-directional (10) or two-dimensional (2D) network of micropores (ie pores<2 nm in diameter)
containing mesopores (pores with diameters in the range 2-50 nm) connected to the microporores, the mesopores being characterized by:
   an aspect ratio (length to width) higher than 2
   a ratio of the volume of the intracrystalline mesopores to the volume of the micropores in the range 0.2 to 2
   an intracrystalline mesoporous volume (“Intra Meso”) that is equal to or higher than 0.02 cm3/g
   and an orientation of the mesopores in the direction of the micropores
      is obtained when following the general preparation method as described in example 1 and varying different parameters such as sodium carbonate concentration, hydrothermal synthesis temperature and duration, and starting material (FIG. 18).

The mesopore sizes and their relative proportions have been determined for three different concentrations of Na₂CO₃ (0.5M, 1.25M and 2M) in the (100) and (010) directions as reported below:
in direction (100);

| | 1-5 nm | | | | | | |
|---|---|---|---|---|---|---|---|
| | 1-2 | 2-3 | 3-4 | 4-5 | Total/ | 5-10 | >10 |
| | nm | nm | nm | nm | nm | nm | nm |
| 0.5M | 91% | 1% | 1% | 2% | 95% | 5% | 0 |
| 1.25M | 21% | 38% | 12% | 23% | 94% | 6% | 0 |
| 2.0M | 0 | 3% | 9% | 23% | 35% | 44% | 21% |

in direction (010):

| | Mesopore size | | | | |
|---|---|---|---|---|---|
| | 1-10 nm | 11-20 nm | 21-30 nm | 31-40 nm | >40 nm |
| 0.5M | 11% | 55% | 26% | 6% | 2% |
| 1.25M | 1% | 31% | 40% | 18% | 10% |
| 2.0M | 0 | 19% | 47% | 21% | 13% |

### Example 8 [Comparative Example] (DeFER1/NaOH-0.05-130-3)

0.83 grams of FER1 (NaKFER, Si/Al=9.2) were used as the parent zeolite, which were treated in 0.05 mol/L of NaOH solution (25 mL, solid/solution=0.0334 g/mL) under the conditions of example 1. The same treatment was performed at 130° C. for 3 days and led to the sample DeFER1/NaOH-0.05-130-3.

| Example 8 | Yield/% | Si/Al | Na/Al | K/Al | (Na + K)/Al |
|---|---|---|---|---|---|
| DeFER1/NaOH- | 85 | 8.5 | 0.16 | 0.67 | 0.83 |
| 0.05-130-3 | | | | | |

| | BET | Pore volume (cm³/g) | | | | | |
|---|---|---|---|---|---|---|---|
| | surface | | | | | | Intra. |
| | area | To- | | Inter. | Intra. | Meso/ | Meso/ |
| Example 8 | (m²/g) | tal | Micro | Meso | Meso | Micro | Micro |
| H-DeFER1/ | 412 | 0.17 | 0.15 | 0.01 | 0.01 | 0.13 | 0.07 |
| NaOH-0.05- | | | | | | | |
| 130-3 | | | | | | | |

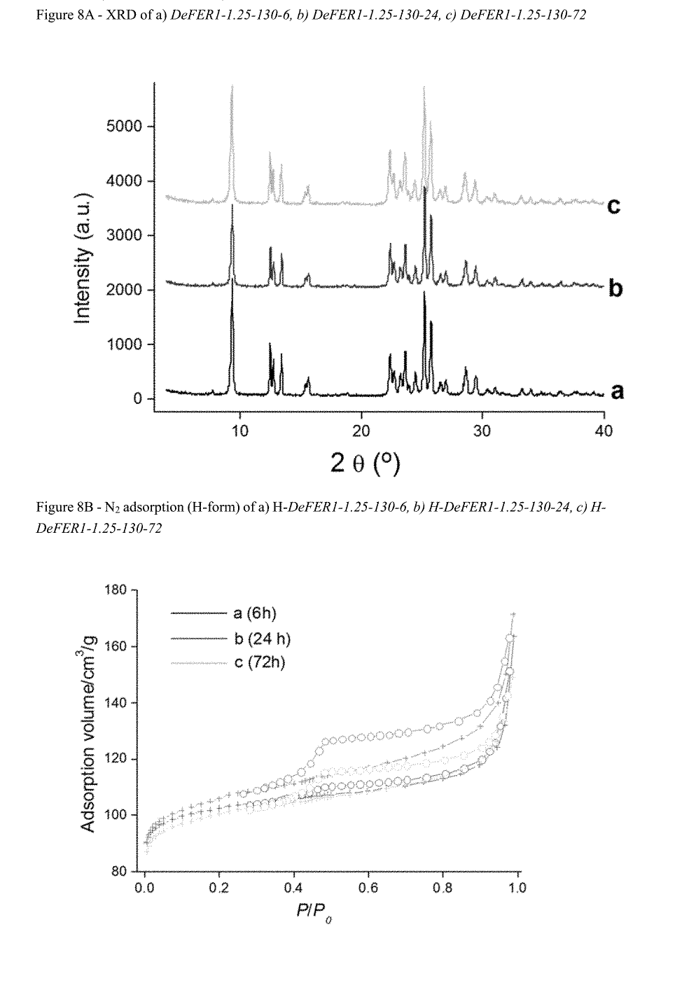

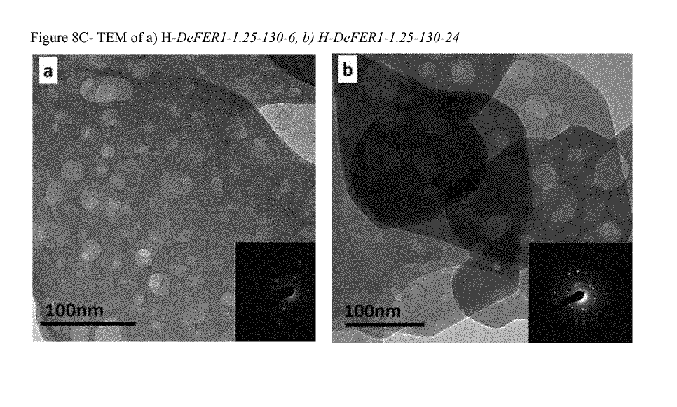

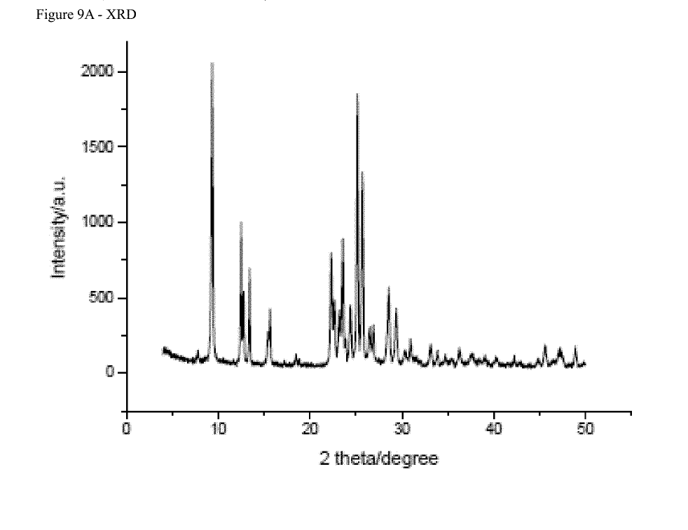

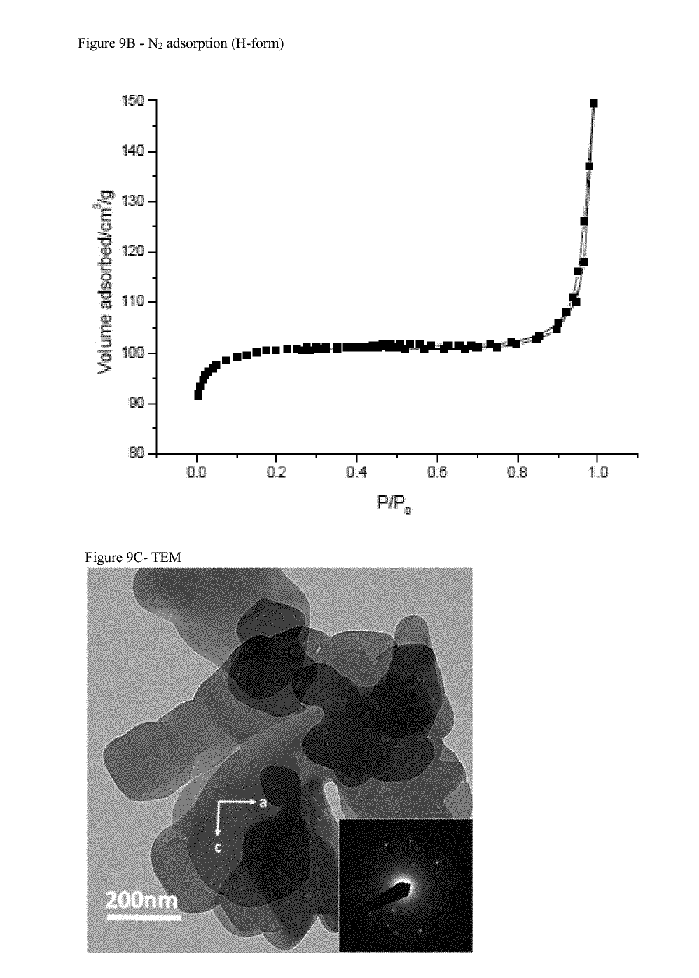

FIG. 9 and above data show that the obtained material does not correspond to the one we claim.

### Example 9 [According to the Invention] (DeFER1/NaOH-0.25-130-3)

0.83 grams of FER1 (NaKFER, Si/Al=9.2) were used as the parent zeolite, which were treated in 0.25 mol/L of NaOH solution (25 mL, solid/solution=0.0334 g/mL) under the conditions of example 1. The same treatment was performed at 130° C. for 3 days and led to the sample DeFER1/NaOH-0.25-130-3.

| Example 9 | Yield/% | Si/Al | Na/Al | K/Al | (Na + K)/Al |
|---|---|---|---|---|---|
| DeFER1/NaOH- | 66 | 5.9 | 0.27 | 0.53 | 0.80 |
| 0.25-130-3 | | | | | |

| | BET | | | | | | |
|---|---|---|---|---|---|---|---|
| | surface | Pore volume (cm³/g) | | | | | |
| | area | To- | | Inter. | Intra. | Meso/ | Intra. |
| Example 9 | (m²/g) | tal | Micro | Meso | Meso | Micro | MesoMicro |
| H-DeFER1/ | 407 | 0.27 | 0.15 | 0.02 | 0.10 | 0.8 | 0.67 |
| NaOH-0.25- | | | | | | | |
| 130-3 | | | | | | | |

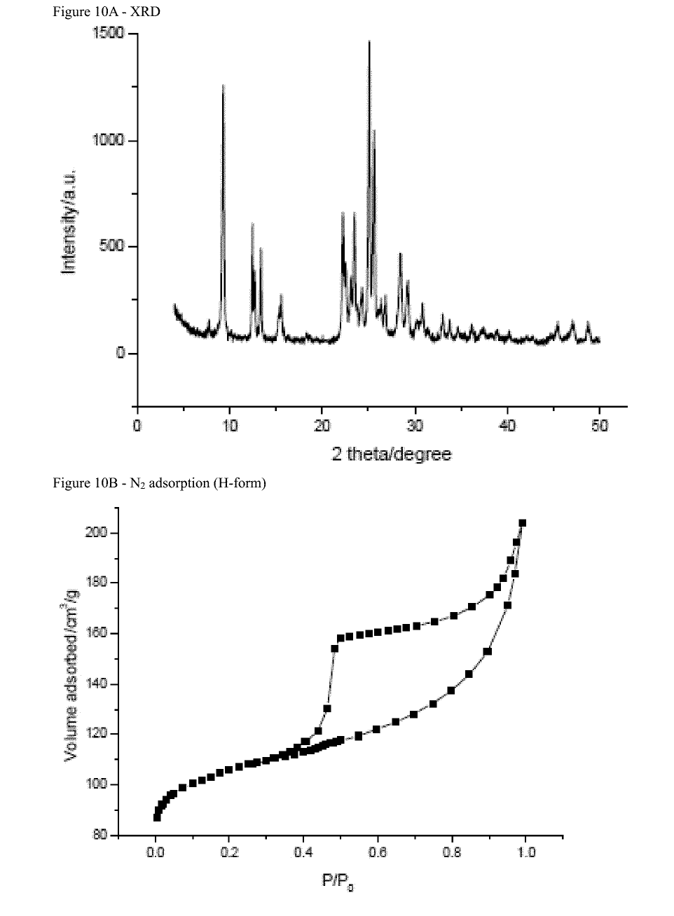

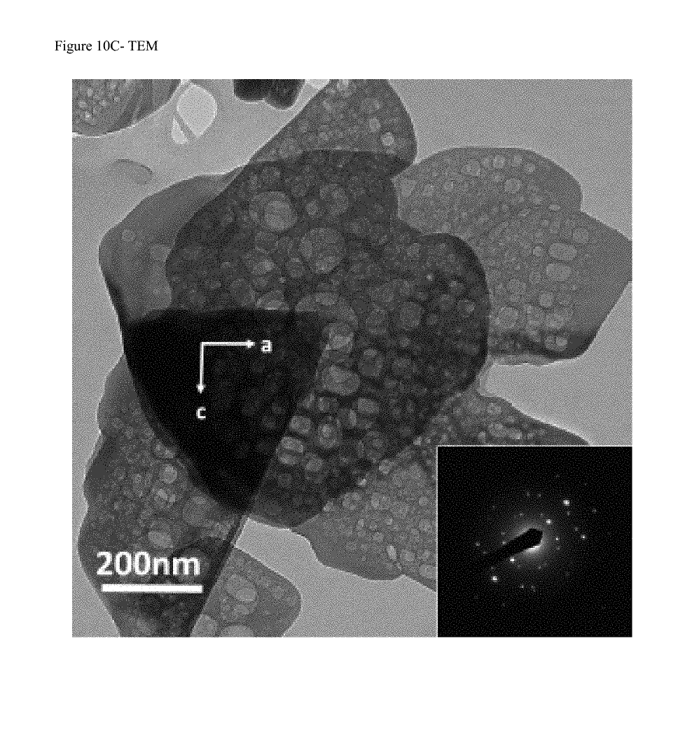

FIG. 10 show that with a NaOH concentration of 0.25 mol/L and a reaction temperature of 130° C., the obtained material corresponds to the one we claim

### Example 10 [Comparative Example] (DeFER1/NaOH-0.50-130-3)

0.83 grams of FER1 (NaKFER, Si/Al=9.2) were used as the parent zeolite, which were treated in 0.50 mol/L of NaOH solution (25 mL, solid/solution=0.0334 g/mL) under the conditions of example 1. The same treatment was performed at 130° C. for 3 days and led to the sample FER1/NaOH-0.50-130-3.

| | Yield/ | | | | (Na + K)/ |
|---|---|---|---|---|---|
| Example 10 | % | Si/Al | Na/Al | K/Al | Al |
| DeFER1/NaOH- | 38 | 2.9 | 0.38 | 0.47 | 0.85 |
| 0.50-130-3 | | | | | |

| | | Pore volume (cm³/g) | | | |
|---|---|---|---|---|---|
| | BET surface area | | | Inter. | Infra. |
| Example 10 | (m²/g) | Total | Micro | Meso | Meso |
| H- | 3 | 0 | 0 | 0 | 0 |
| DeFER1/NaOH- | | | | | |
| 0.50-130-3 | | | | | |

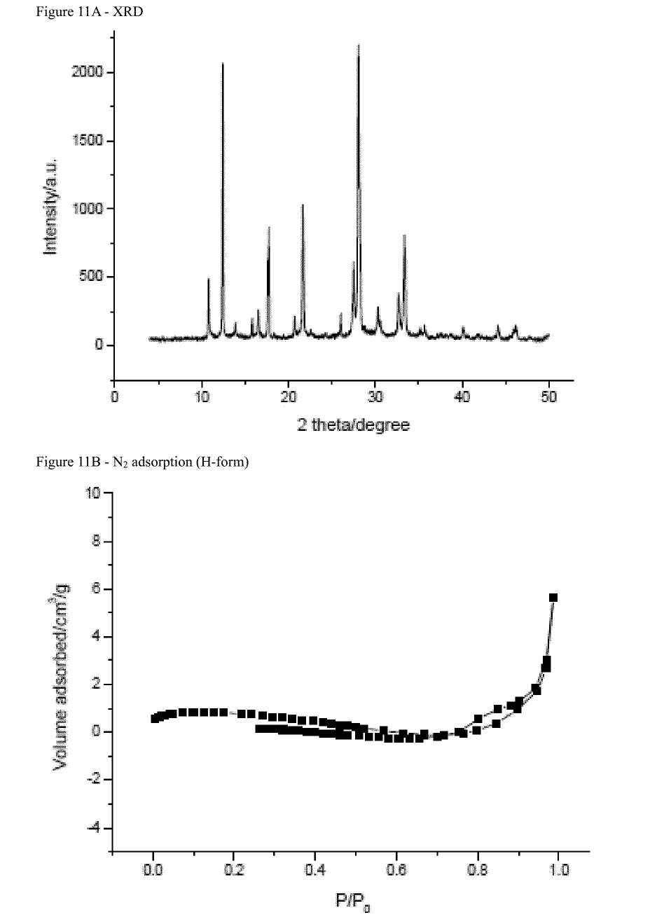

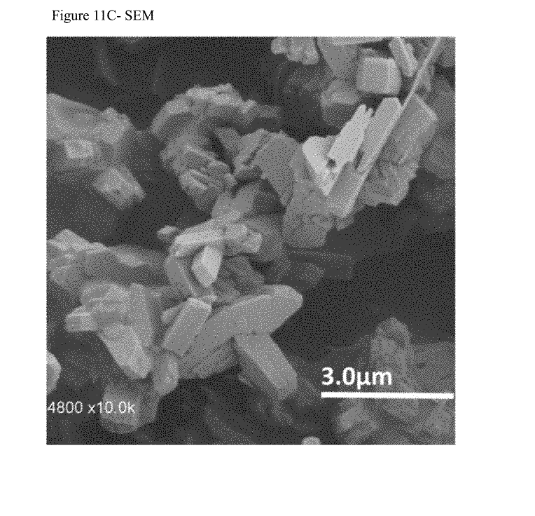

FIG. 11 show that the obtained material does not correspond to the one we claim: the solid recovered shows the presence of GIS phase.

### Example 11 [Comparative Example]

0.83 grams of FER1 (NaKFER, Si/Al=9.2) were used as the parent zeolite, which were treated in 0.5 mol/L of NaOH solution (25 mL, solid/solution=0.0334 g/mL) and then stirred for 30 min. The suspension was hydrothermally treated at 80° C. in a Teflon-lined stainless autoclave for 3 hours. After cooling the autoclave to room temperature, the solid was filtered under vacuum and washed with de-ionized water repeatedly until pH=7. Finally the product was dried overnight at 80° C. to get the sample of DeFER1/NaOH-0.5-80-3 h.

The as-synthesized DeFER1/NaOH-0.5-80-3 h sample was ion-exchanged in 1.0 mol/L NH4NO3 solution for 6 h at room temperature, after dryness, the sample was calcined in air flow (100 mL/min) in a tubular furnace at 550° C. for 8 h, and the sample denoted as H-DeFER1/NaOH-0.5-80-3 h was obtained.

| | BET | | | | | | |
|---|---|---|---|---|---|---|---|
| | surface | Pore volume (cm³/g) | | | | | |
| | area | To- | | Inter. | Intra. | Meso/ | IntraMeso/ |
| Example 11 | (m²/g) | tal | Micro | Meso | Meso | Micro | Micro |
| H-DeFER1/ | 417 | 0.19 | 0.15 | 0.03 | 0.01 | 0.27 | 0.07 |
| NaOH-0.5- | | | | | | | |
| 80-3 h | | | | | | | |

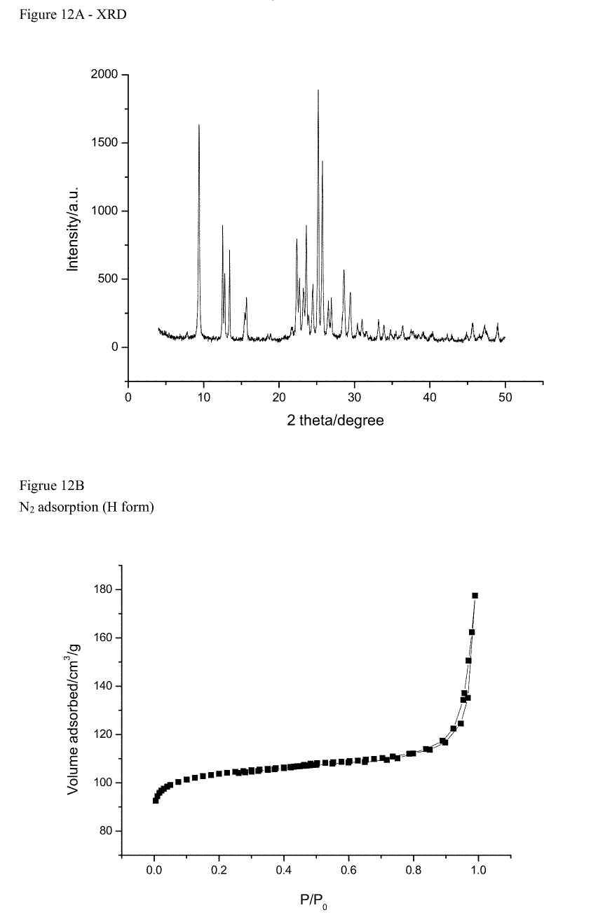

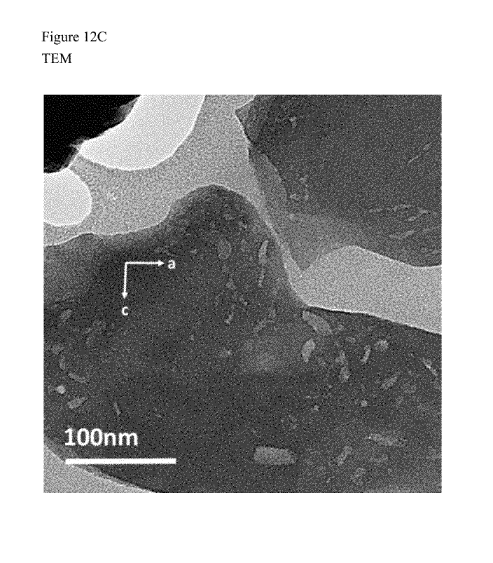

The reaction of FER1 in the presence of NaOH allowed to generate some intracrystalline mesopores (FIG. 12) but due to the too low temperature (80° C.), it is not possible to recover a mesoporous FER corresponding to the one we claim.

### Example 12 [According to the Invention]

1.67 g of commercial NaKFER (FER1, Si/Al=9.2) was first mixed with 50 mL of 1.25 mol/L Na₂CO₃ solution, and then stirred for 30 min. To this mixture cetyltrimethylammonium bromide (CTAB) was added in order to obtain a mass ratio CTAB/FER1 equal to 0.5. The mixture was then treated according to the same procedure as in example 1 to yield DeFER1-1.25-130/3-CTAB and H-DeFER1-1.25-130/3-CTAB.

| Example 12 | Yield/% | Si/Al | Na/Al | K/Al | (Na + K)/Al |
|---|---|---|---|---|---|
| DeFER1-1.25- | 83.9 | 9.3 | 0.36 | 0.56 | 0.92 |
| 130/3-CTAB | | | | | |

| | BET | Pore volume (cm³/g) | | | | | |
|---|---|---|---|---|---|---|---|
| | surface | | | | | | Intra. |
| | area | To- | | Inter. | Intra. | Meso/ | Meso/ |
| Example 12 | (m²/g) | tal | Micro | Meso | Meso | Micro | Micro |
| H-DeFER1- | 505 | 0.39 | 0.12 | 0.09 | 0.18 | 2.3 | 1.5 |
| 1.25-130/ | | | | | | | |
| 3-CTAB | | | | | | | |

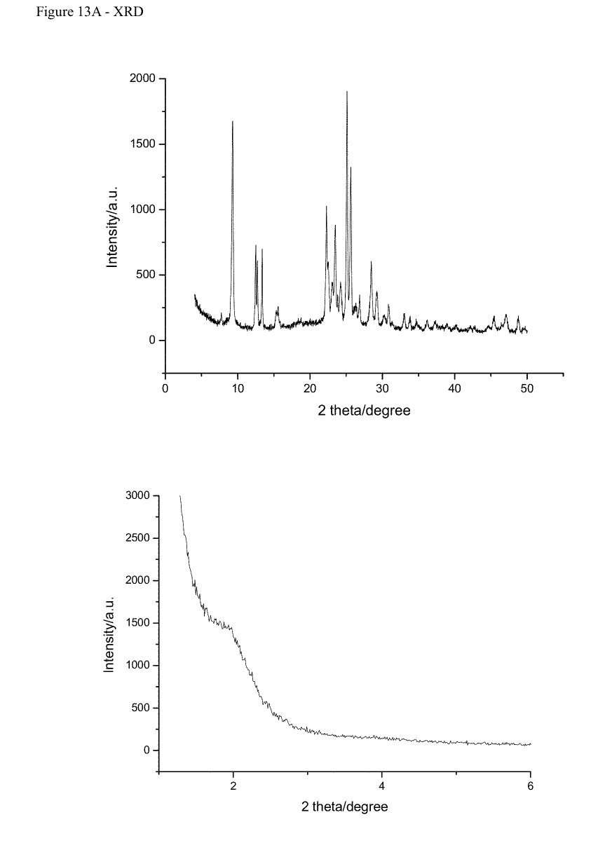

This example shows that the addition of a surfactant to the reaction mixtures allows a remarkable increase of the volume of the intracystalline occluded mesopores (Table 6, FIG. 13B). Moreover the material is highly crystalline and features a long range ordering of the mesopores as demonstrated by the correlation peak at low 2 theta values in the XRD diffractogram (FIG. 13A). The high degree or order of the mesopores is apparent from the TEM images (FIG. 13C). Seen in the (010) direction (i.e. normal to the 10 MR channels of the zeolite) their size is in the range 30-50 nm while the width measured in the (100) direction (i.e. parallel to the 10 MR channel) is in the range 10-20 nm.

### Example 13 [Comparative Example] (FER1/NaOH-0.05-130-3-CTAB)

0.83 grams of FER1 (NaKFER, Si/Al=9.2) were used as the parent zeolite, which were treated in 0.05 mol/L of NaOH solution (25 mL, solid/solution=0.0334 g/mL) under the conditions of example 1, to this mixture cetyltrimethylammonium bromide (CTAB) was added in order to obtain a mass ratio CTAB/FER1 equal to 0.5. The same treatment was performed at 130° C. for 3 days and led to the sample FER1/NaOH-0.05-130-3-CTAB.

| Example 13 | Yield/% | Si/Al | Na/Al | K/Al | (Na + K)/Al |
|---|---|---|---|---|---|
| FER1/ | 94 | 7.9 | 0.13 | 0.69 | 0.82 |
| NaOH-0.05- | | | | | |
| 130-3-CTAB | | | | | |

| | BET | Pore volume (cm³/g) | | | | | |
|---|---|---|---|---|---|---|---|
| | surface | | | | | | Intra |
| | area | To- | | Inter. | Intra. | Meso/ | Meso/ |
| Example 13 | (m²/g) | tal | Micro | Meso | Meso | Micro | Micro |
| FER1/ | 390 | 0.18 | 0.14 | 0.03 | 0.01 | 0.29 | 0.07 |
| NaOH-0.05- | | | | | | | |
| 130-3- | | | | | | | |
| CTAB | | | | | | | |

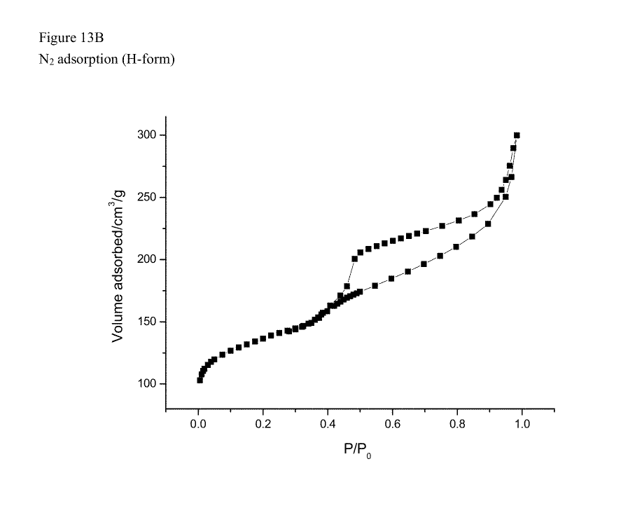

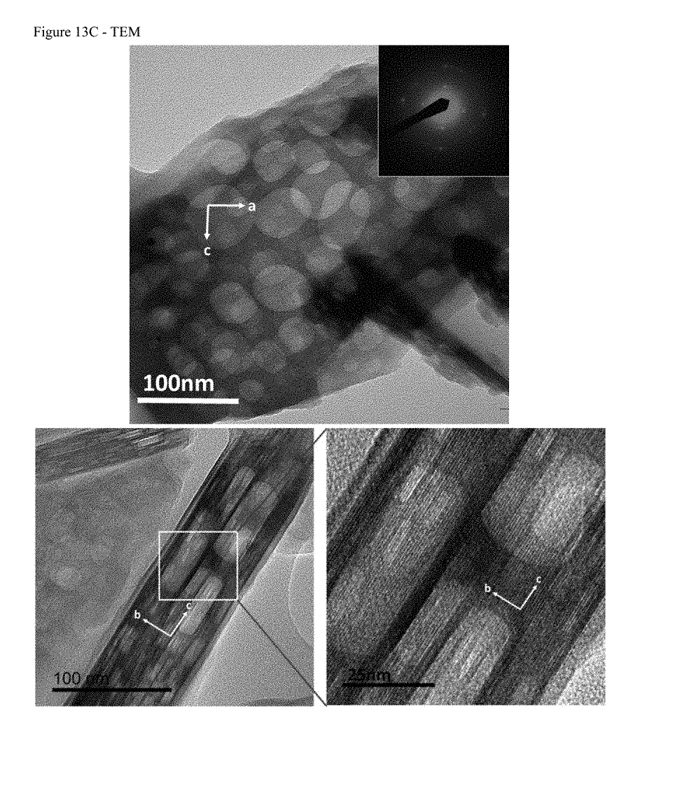

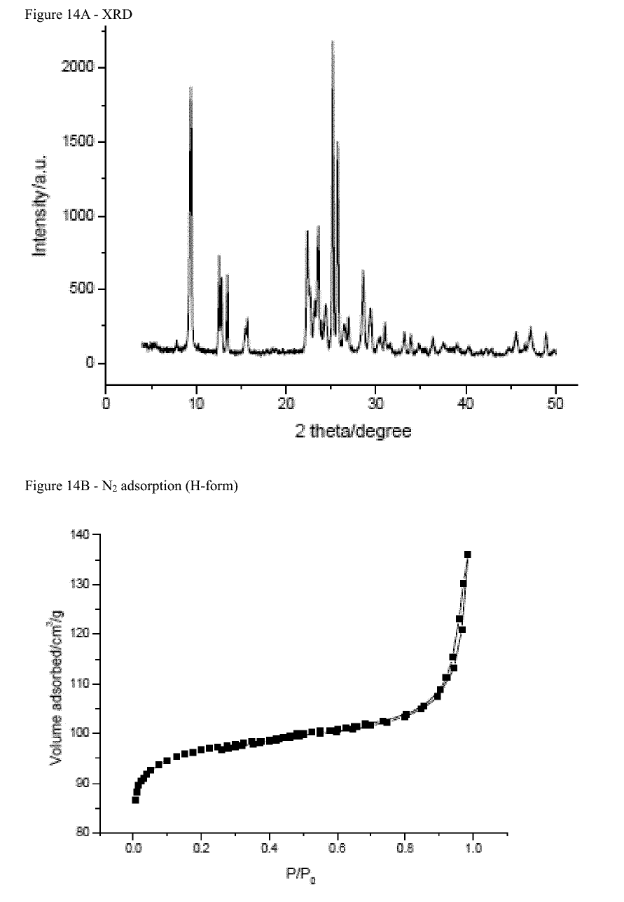

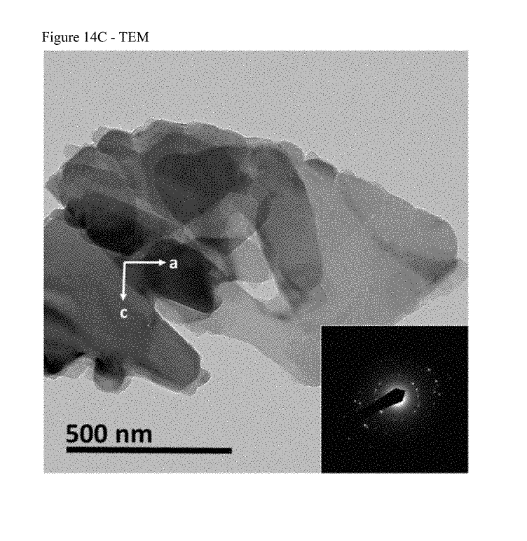

FIG. 14 show that the material obtained does not correspond to the one we claim.

### Example 14 [According to the Invention] (FER1/NaOH-0.25-130-3-CTAB)

0.83 grams of FER1 (NaKFER, Si/Al=9.2) were used as the parent zeolite, which were treated in 0.25 mol/L of NaOH solution (25 mL, solid/solution=0.0334 g/mL) under the conditions of example 1, to this mixture cetyltrimethylammonium bromide (CTAB) was added in order to obtain a mass ratio CTAB/FER1 equal to 0.5. The same treatment was performed at 130° C. for 3 days and led to the sample FER1/NaOH-0.25-130-3-CTAB.

| Example 14 | Yield/% | Si/Al | Na/Al | K/Al | (Na + K)/Al |
|---|---|---|---|---|---|
| FER1/NaOH- | 70 | 6.5 | 0.29 | 0.58 | 0.87 |
| 0.25-130- | | | | | |
| 3-CTAB | | | | | |

| | BET | | | | | | |
|---|---|---|---|---|---|---|---|
| | surface | Pore volume (cm³/g) | | | | | |
| | area | To- | | Inter. | Intra. | Meso/ | IntraMeso/ |
| Example 14 | (m²/g) | tal | Micro | Meso | Meso | Micro | Micro |
| FER1/NaOH- | 407 | 0.29 | 0.13 | 0.03 | 0.13 | 1.23 | 0.87 |
| 0.25-130- | | | | | | | |
| 3-CTAB | | | | | | | |

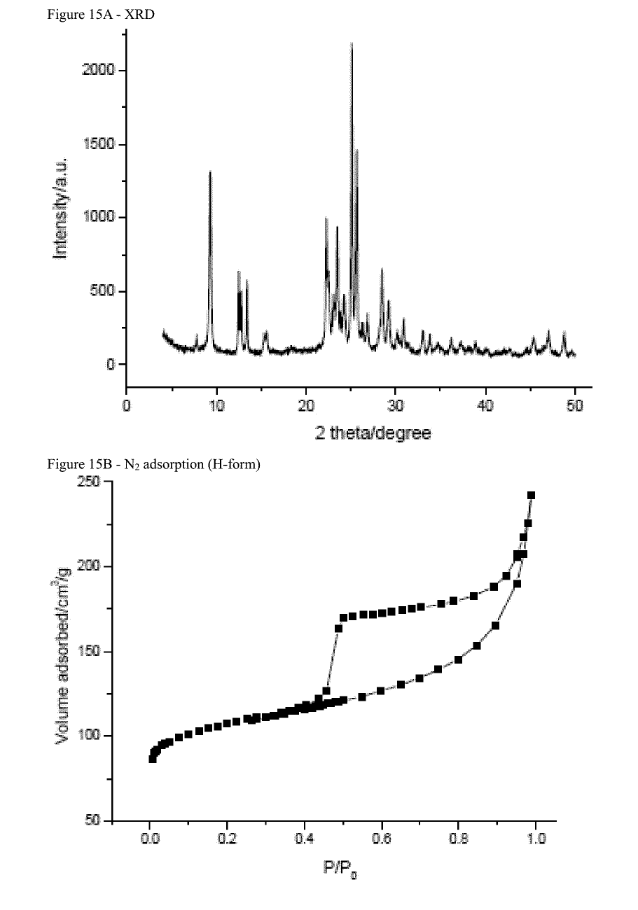

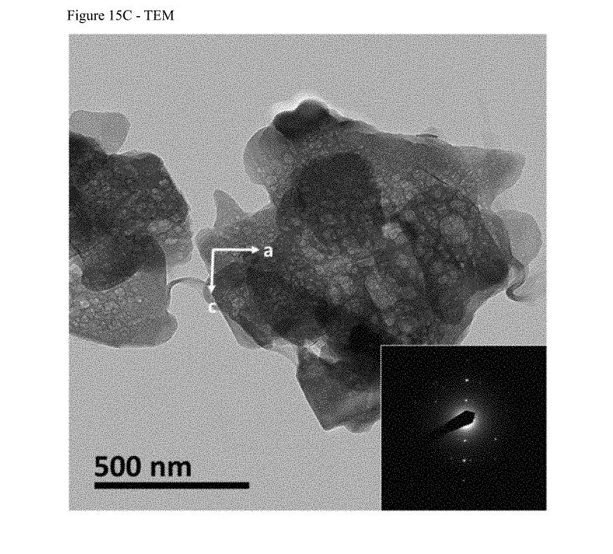

FIG. 15 show that with a NaOH concentration of 0.25 mol/L, the presence of CTAB and a reaction temperature of 130° C. the material obtained corresponds to the one we claim

### Example 15 (FER1/NaOH-0.50-130-3-CTAB)

0.83 grams of FER1 (NaKFER, Si/Al=9.2) were used as the parent zeolite, which were treated in 0.50 mol/L of NaOH solution (25 mL, solid/solution=0.0334 g/mL) under the conditions of example 1, to this mixture cetyltrimethylammonium bromide (CTAB) was added in order to obtain a mass ratio CTAB/FER1 equal to 0.5. The same treatment was performed at 130° C. for 3 days and led to the sample FER1/NaOH-0.50-130-3-CTAB.

| Example 15 | Yield/% | Si/Al | Na/Al | K/Al | (Na + K)/Al |
|---|---|---|---|---|---|
| FER1/NaOH- | 75 | 2.2 | 0.21 | 0.28 | 0.49 |
| 0.50-130- | | | | | |
| 3-CTAB | | | | | |

| | BET | | | | | | |
|---|---|---|---|---|---|---|---|
| | surface | Pore volume (cm³/g) | | | | | |
| | area | To- | | Inter. | Intra. | Meso/ | IntraMeso/ |
| Example 15 | (m²/g) | tal | Micro | Meso | Meso | Micro | Micro |
| FER1/NaOH- | 197 | 0.25 | 0.07 | 0.1 | 0.08 | 2.6 | 1.1 |
| 0.50-130- | | | | | | | |
| 3-CTAB | | | | | | | |

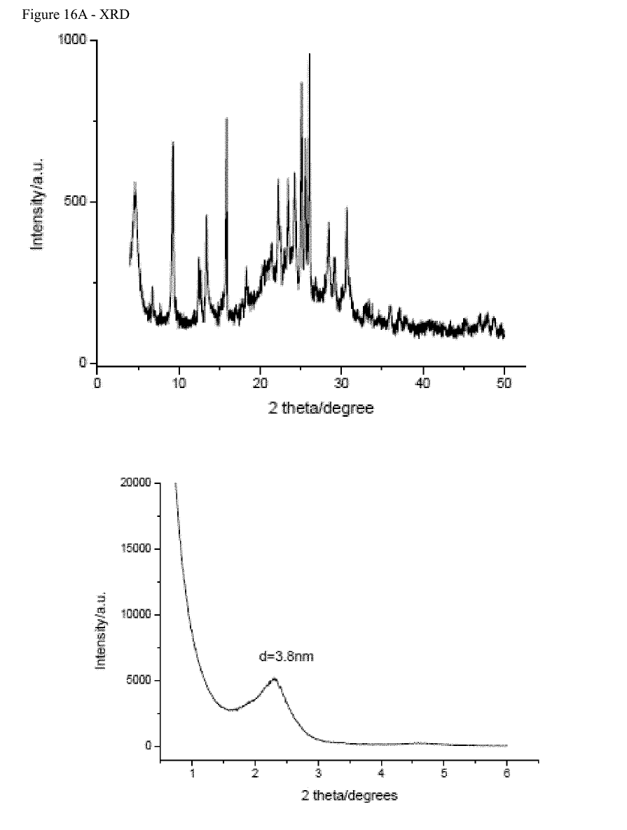

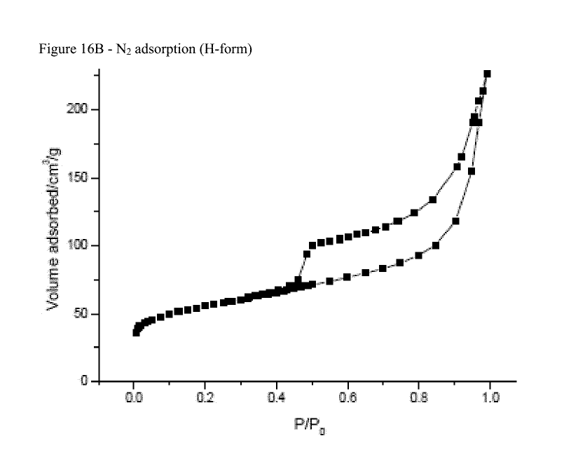

FIG. 16 shows that by increasing NaOH concentration up to 0.5M in presence of a surfactant such as CTAB, the material transformation is enhanced to such an extent that a mixture of lamellar-phase and FER crystalline phase containing occluded and oriented mesopores is obtained.

Catalytic Performances: Oligomerization of Pentenes:

The performances of the catalysts FER2 (parent zeolite—example 0) and DeFER1-1.25-130/3 (desilicated FER—example 1) were evaluated in oligomerization of a model feed consisting in a mixture of n-heptane (nC7) and 1-pentene (105=).

The FER samples (parent and desilicated) were pressed into wafers, crushed and sieved to obtain particles with diameters of 150-250 μm. Catalytic reactions were conducted down-flow in a tubular fixed-bed down-flow reactor (6 mm internal diameter) loaded with 1 g of catalyst.

The catalyst was supported by a porous disk (60 μm) and the dead volume was filled with quartz particles of 200-400 μm in size. The catalyst temperature was monitored with a thermocouple placed inside the bed. The catalyst, previously activated in flowing air (100 mL/min at 550° C. for 8 h) was loaded into the reactor and dehydrated at 180° C. for three hours in flowing air.

Pure n-heptane was then fed to the system using a HPLC pump (Gilson) until the operating pressure (50 barg) was achieved. The n-heptane flow was then shifted to the reagent feedstock, consisting of a 50/50 mixture of pent-1-ene and n-heptane (both from Sigma-Aldrich, 99% purity, WHSV: 0.5-2 h-1).

Reactor pressure was regulated using an Equilibar back-pressure regulator.

The catalytic tests were performed under the following operating conditions:

50 barg/WHSV(Weight Hourly Space Velocity) varying from 0.5 to 2 h⁻¹/Temperature varying from 150 up to 200° C.

Analysis of the products is performed by using an on-line gas chromatography (Agilent 6850, using a capillary column DB 2887 (100% Diméthylpolysiloxane, 10 m, 0.53 mm, 3 μm).

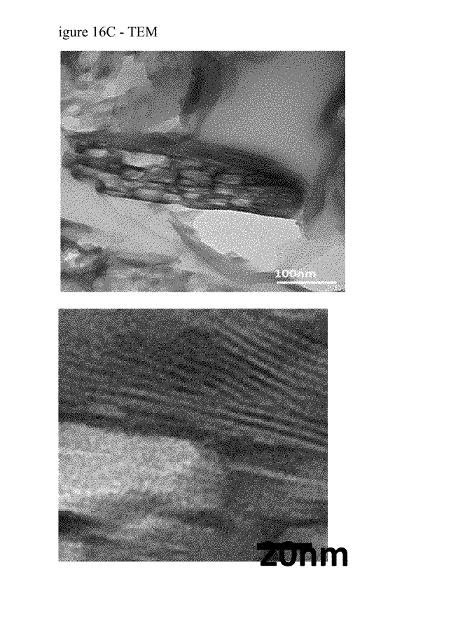

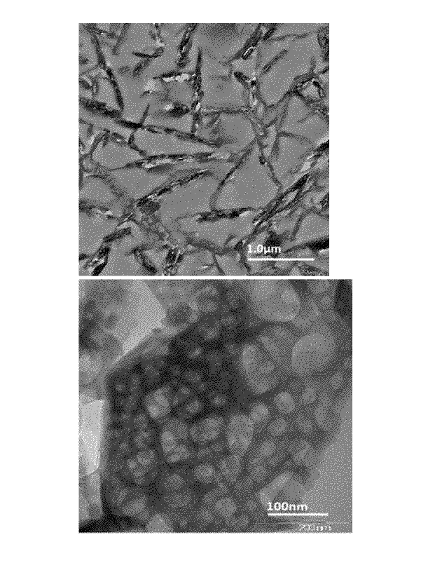

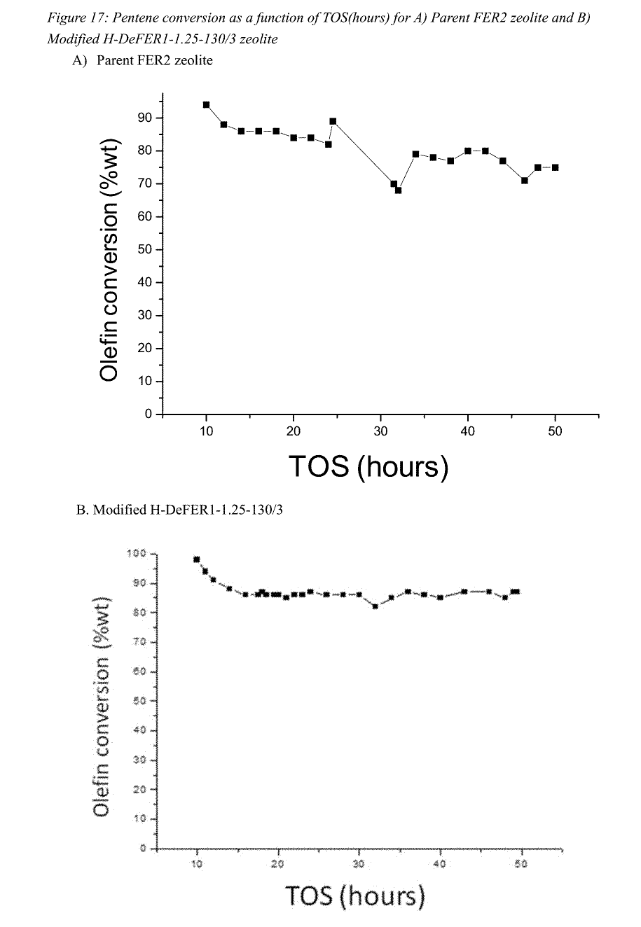

The performances of parent zeolite (catalyst FER2) and of alkalinetreated sample (catalyst H-DeFER1-1.25-130/3 example 1) are presented in FIG. 17 and table 6.

FIG. 17 show that when the oligomerization of pentene is conducted on FER2 sample, the conversion of pentene deactivates slowly from 95% wt down to 75% wt within only 50 hours TOS (TOS=Time On Stream). The main products formed are dimers (C10), followed by trimers (C15) and heavier oligomers (C20+). The micropores limit the diffusion of the heavy oligomers, which remain stuck within the microporous structure of the material, leading to its progressive deactivation.

By applying the desilication treatment to parent zeolite (DeFER1-1.25-130/3), the pentene conversion stabilizes at around 85% wt after 15 hours of stabilization period, while in the meantime, the formation of heavier oligomers becomes more favorable. The introduction of the mesoporosity within the FER structure, even if occluded, allows a better diffusion of the heavy molecules, and so the formation of higher amount of larger oligomers.

The pentene conversion and oligomers distribution for parent FER zeolite (FER2) and the modified FER (H-DeFER1-1.25-130/3) are as follows: with parent FER zeolite (FER2), the pentene conversion decreases from 90% (at 10 h of time on stream) down to 76% after 47 hours of TOS, while it remains stable at 86% from 15 h to 47 h of TOS in the case of the modified FER (H-DeFER1-1.25-130/3).

Selectivities vary as follows:
with parent FER zeolite (FER2): fraction of C10 oligomers increases from 40% to 65% from 10 to 47 hours of TOS, while the C15+C20 fraction decreases from 60 to 35% at the same time.
with the modified FER (H-DeFER1-1.25-130/3), fraction of C10 oligomers remains stable at about 37% from 10 to 47 hours of TOS, while the C15+C20+ fraction remains stable at about 63%.

1.-15. (canceled)

16. A mesoporous zeolitic material possessing an ordered mono-dimensional (1D) or two-dimensional (2D) network of micropores, wherein the micropores are less than 2 nm in diameter, the material comprising mesopores with diameters in the range 2-50 nm connected to the micropores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores.

17. A mesoporous zeolitic material according to claim 16, which network of micropores has a geometry consistent with one of MTT (ZSM-23), TON (ZSM-22, Theta-1, NU-10), EUO (ZSM-50, EU-1), FER (ferrierite, FU-9, ZSM-35), MWW (MCM-22, PSH-3, ITQ-1, MCM-49), MFS (ZSM-57), and ZSM-48.

18. A process for preparing the mesoporous zeolitic material possessing an ordered mono-dimensional (1D) or two-dimensional (2D) network of micropores, wherein the micropores are less than 2 nm in diameter, the material comprising mesopores with diameters in the range 2-50 nm connected to the micropores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores, comprising the following steps:
i) contacting a parent zeolitic material with a basic aqueous solution containing at least one weak base having a pKa of at least 7 and at most 14 in water, at a concentration ranging from 0.5M to 3M, to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., under pressure from 2 to 20 bara, the pressure being autogenously generated,
iii) filtering off the zeolite obtained at step (ii) and washing it with a polar solvent, to obtain a washed zeolite,
iv) optionally drying the washed zeolite,
v) placing the washed and optionally dried zeolite in contact, in a solution, especially an aqueous solution, of NH4NO3, especially at a concentration ranging from 0.01 to 0.5 M,
vi) washing the zeolite obtained at step (v) with distilled water to a neutral pH,
vii) calcining the zeolite obtained at step (vi), and recovering the mesoporous zeolitic material.

19. A process for preparing the mesoporous zeolitic material possessing an ordered mono-dimensional (1D) or two-dimensional (2D) network of micropores, wherein the micropores are less than 2 nm in diameter, the material comprising mesopores with diameters in the range 2-50 nm connected to the micropores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores, comprising the following steps:
i) contacting a parent zeolitic material with a basic aqueous solution containing at least a strong base that is totally dissociated in water at a concentration ranging from 0.2M to 0.3M, to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., under pressure from 2 to 20 bara, the pressure being autogenously generated,
iii) filtering off the zeolite obtained at step (ii) and washing it with a polar solvent, to obtain a washed zeolite,
iv) optionally drying the washed zeolite,
v) placing the washed and optionally dried zeolite in contact, in a solution, especially an aqueous solution, of NH₄NO₃, at a concentration ranging from 0.01 to 0.5 M,
vi) washing the zeolite obtained at step (v) with distilled water to a neutral pH,
vii) calcining the zeolite obtained at step (vi), and recovering the mesoporous zeolitic material.

20. A process for preparing the mesoporous zeolitic material possessing an ordered mono-dimensional (1D) or two-dimensional (2D) network of micropores, wherein the micropores are less than 2 nm in diameter, the material comprising mesopores with diameters in the range 2-50 nm connected to the micropores, the mesopores being characterized by an aspect ratio (length to width) higher than 2, a ratio of the volume of the intracrystalline mesopores to the volume of the micropores in the range 0.1 to 2 and an orientation of the mesopores in the direction of the micropores, comprising the following steps:
i) contacting a parent zeolitic material with a basic aqueous solution containing at least one weak base having a pKa ranging from 7 to 9 at a concentration ranging from 1M to 2M, and/or a strong base that is totally dissociated in water at a concentration ranging from 0.2M to 0.5M in the presence of a mesopore organic structure directing agent, to obtain a first composition,
ii) heating said first composition in a vessel at a temperature sufficient to increase the pressure above the atmospheric pressure in said vessel or at a pressure of at least 2 bara and at a temperature of at least 100° C. or at a temperature from 100 to 150° C., under pressure from 2 to 20 bara, the pressure being autogenously generated
iii) filtering off the zeolite obtained at step (ii) and washing it with a solvent, especially a polar solvent, for example pure distilled water, to obtain a washed zeolite,
iv) optionally drying the washed zeolite,
v) placing the washed and optionally dried zeolite in contact, in a solution, especially an aqueous solution, of NH₄NO₃, at a concentration ranging from 0.01 to 0.5 M,
vi) washing the zeolite obtained at step (v) with distilled water to a neutral pH,
vii) calcining the zeolite obtained, at step (vi) and recovering the mesoporous zeolitic material.

21. A process according to claim 20, wherein the mesopore structure directing agent is a surfactant.

22. A process according to claim 20, wherein the mesopore structure directing agent is is cetyltrimethylammonium bromide (CTAB).

23. A process according to claim 20, wherein the mesopore structure directing agent is a recyclable surfactant able to generate a micellization upon the effect of the variation of a physico-chemical parameter (pH, temperature, ionic strength).

24. A process according to claim 20, in wherein the mesopore structure directing agent contains an oligomeric or polymeric chain bearing at least one ionic function and rendered amphiphilic upon the effect of the variation of a physico-chemical parameter, the physico-chemical parameter selected from among pH, temperature and ionic strength, wherein the mesopore structure directing agent is selected among:
a statistical copolymer of ethylene and propylene functionalized by a quaternary ammonium salt, such as Jeffamines, the molecular size of which varying from 140 to 5000 g/mol and the ethylene oxide/propylene oxide molar ratio of which varying from 0.01 to 5, said Jeffamines being quaternized on their primary amine wherein the amino group of the mesopore-templating agent is quaternized; or
is a Jeffamine selected among Jeffamine M600 and Jeffamine M2005 wherein the amino group of the mesopore-templating agent is quaternized.

25. A process according to claim 18, wherein the alkaline metal in the alkaline metal carbonate is selected among ammonium, sodium and potassium, and/or their mixtures.

26. A process according to claim 19, wherein the alkaline hydroxide is selected among ammonium, sodium and potassium hydroxides, and their mixtures.

27. A process according to claim 18, wherein the parent zeolitic material is a mono-dimensional (1D) micropore architecture zeolite selected from the groups MTT (ZSM-23), TON (ZSM-22, Theta-1, NU-10), EUO (ZSM-50, EU-1).

28. A process according to claim 18, wherein the parent zeolitic material is a two-dimensional (2D) inter-connecting micropore architecture zeolite selected from the groups FER (ferrierite, FU-9, ZSM-35), MWW (MCM-22, PSH-3, ITQ-1, MCM-49), MFS (ZSM-57), ZSM-48.

29. A process according to claim 28, wherein the parent zeolitic material belongs to the FER group.

30. The use of a material according to claim 16 as a refining or a petrochemical catalyst.
